(19) United States
(12) Patent Application Publication
(10) Pub. No.: US 2023/0093253 A1
(43) Pub. Date: Mar. 23, 2023

(54) AUTOMATICALLY IDENTIFYING FAILURE SOURCES IN NUCLEOTIDE SEQUENCING FROM BASE-CALL-ERROR PATTERNS

(71) Applicants: Illumina, Inc., San Diego, CA (US); Illumina Software, Inc., San Diego, CA (US)

(72) Inventors: Thomas Gros, Encinitas, CA (US); Zoey Wei Chesny, San Diego, CA (US)

(21) Appl. No.: 17/821,410

(22) Filed: Aug. 22, 2022

Related U.S. Application Data
(60) Provisional application No. 63/245,639, filed on Sep. 17, 2021.

(51) Int. Cl. G16B 30/00; G06N 3/12

(52) U.S. Cl. CPC G16B 30/00; G06N 3/123

(57) ABSTRACT

Methods, systems, and non-transitory computer readable media are disclosed for accurately and efficiently identifying base-call-error scars or patterns from sequencing data to determine failure sources that contribute to the base-call-error scars or patterns. For example, the disclosed system can utilize a reference genome to determine nucleotide-specific errors within a run of a sequencing pipeline. Based on the co-occurrence of different nucleotide-specific errors, the disclosed system can determine a base-call-error scar. The disclosed system can further determine one or more sample error scars from sample sequencing runs that correlate to the base-call-error scar. Based on the correlation and by utilizing a statistical model, the disclosed system can identify failure sources contributing to the nucleotide-specific errors within the base-call-error scar.

### CROSS-REFERENCE TO RELATED APPLICATIONS

This. This application claims the benefit of, and priority to, U.S. Provisional Application No. 63/245,639, entitled “AUTOMATICALLY IDENTIFYING FAILURE SOURCES IN NUCLEOTIDE SEQUENCING FROM BASE-CALL-ERROR PATTERNS,” filed Sep. 17, 2021, the contents of which are hereby incorporated by reference in their entirety.

### BACKGROUND

In recent years, biotechnology firms and research institutions have improved hardware and software platforms to determine a sequence of nucleotide bases or a whole genome. For instance, some existing nucleic-acid-sequencing platforms determine individual nucleotide bases within sequences by using existing Sanger sequencing or sequencing-by-synthesis (SBS). When using SBS, existing platforms can monitor tens of thousands or more oligonucleotides being synthesized in parallel to determine nucleotide-base calls. For instance, a camera in SBS platforms can capture images of irradiated fluorescent tags from nucleotide bases incorporated into to such oligonucleotides. After capturing images, existing SBS platforms send base-call data (or image data) to a computing device with sequencing-data-analysis software that aligns nucleotide reads with a reference genome. Based on the aligned nucleotide-fragment reads, existing SBS platforms can determine nucleotide-base calls for genomic regions and identify variants within a sample's nucleic-acid sequence.

Despite advances in sequencing, existing nucleotide-base-sequencing platforms and sequencing-data-analysis software (together and hereinafter, existing sequencing systems) frequently determine incorrect nucleotide-base calls at positions throughout a genome or during a sequencing run, but cannot accurately or efficiently detect systemic or random causes of such incorrect nucleotide-base calls. Indeed, existing sequencing systems can determine incorrect base calls—or slow or stop the yield of base calls in sequencing runs—because of complex-hardware failures, faulty reagents interacting with each other or with nucleotides, or sophisticated software that incorrectly analyze nucleotide reads or other base-call data. While some existing sequencing systems include sensors within tubing or other parts of a sequencing machine, such in-machine sensors can only detect a relatively small subset of hardware or reagent failures and can entirely fail to detect software errors. In addition to in-machine sensors, some existing systems utilize software trimming tools to exclude the ends of nucleotide-fragment reads or other parts of input data with lower quality scores. By reducing nucleotide-fragment-read length, however, conventional trimming tools often aggravate coverage bias and thereby introduce other complexities to detecting systemic errors. Further to the point, many conventional error correction tools—such as Bayesian clustering for error correction, Bloom Filter Correction (BFC), Bloom-filter-based Error Correction Solution for High-throughput Sequencing Reads (BLESS), and other tools—are designed to correct common read errors or extend certain reads, but give little to no indication of the underlying cause of such errors. With many potential points of failure in chemistry, machinery, or software, existing sequencing systems frequently cannot accurately pinpoint underlying factors that contribute to data quality or yield of base calls.

In addition to inaccurate or non-existent failure detection, existing sequencing systems often can only detect systemic errors using inefficient or bulky detection sensors or algorithms. For example, existing systems often expend additional processing, computing, storage resources, and time to identify sources of errors correctly or incorrectly in sequencing. Conventional systems often utilize methods and algorithms to analyze a genome and correct errors. Such methods and algorithms are computationally costly. In one example, existing systems utilize Louvian community detection algorithms by analyzing read pairs and generating similarity scores between read pairs. To reduce the computational costs of generating similarity scores for each read pair, some existing systems analyze specific segments of a sequence and must disregard other segments. But calculating similarity scores between each read pair is often both computationally intensive and time intensive. Because existing systems often fail to efficiently identify sources of failure, they often require users to repeat sequencing runs multiple times before successfully identifying an issue.

Beyond computationally intensive error detection, some existing sequencing systems inflexibly address only certain types of errors. Generally, sequencing platforms lack the infrastructure required to identify the broad spectrum of potential failure sources occurrent in existing systems. For example, existing sequencing systems often utilize a Phred algorithm to determine quality scores that estimate a likelihood that an individual base call is incorrect. Even though existing systems can estimate individual base-call errors, they typically cannot identify root causes of such base-call errors. To illustrate, existing systems typically cannot indicate whether a particular error stems from faults in machinery, reagents, chemistry, or software.

These, along with additional problems and issues exist in existing sequencing systems.

### BRIEF SUMMARY

This disclosure describes one or more embodiments of systems, methods, and non-transitory computer readable storage media that solve one or more of the problems described above or provide other advantages over the art. In particular, the disclosed systems can accurately and efficiently identify a base-call-error scar or pattern from the sequencing data of a sequencing pipeline and determine failure sources that contribute to the base-call-error scar or pattern. For instance, the disclosed system can utilize a reference genome to determine nucleotide-specific errors within a sequencing run of a sequencing pipeline. Based on different magnitudes or combinations of nucleotide-specific errors, the disclosed system can further identify a base-call-error scar among the base-call data of the sequencing pipeline. The disclosed system can further analyze data from sample sequencing runs using the same or similar sequencing pipeline and apply a statistical model to identify sample base-call-error scars from the sample sequencing runs that correlate to the base-call-error scar. Based on the correlation between the base-call-error scar from the data of the sequencing pipeline and one or more corresponding sample base-call-error scars, the disclosed system can identify failure sources contributing to the nucleotide-specific errors among the base-call-error scar. For instance, the disclosed system can identify failure sources in hardware, chemistry, or software.

Additional features and advantages of one or more embodiments of the present disclosure will be set forth in the description which follows, and in part will be obvious from the description, or may be learned by the practice of such example embodiments.

### BRIEF DESCRIPTION OF THE DRAWINGS

The detailed description will describe various embodiments with additional specificity and detail through the use of the accompanying drawings, which are summarized below.

FIG. 1 illustrates an environment in which a variation-source-identification system can operate in accordance with one or more embodiments of the present disclosure.

FIG. 2 illustrates an overview diagram of the variation-source-identification system detecting a base-call-error pattern from the sequencing data of a sequencing pipeline and determining a failure source based on the base-call-error pattern in accordance with one or more embodiments of the present disclosure.

FIG. 3 illustrates the variation-source-identification system determining base-call-error rates in accordance with one or more embodiments of the present disclosure.

FIG. 4 illustrates the variation-source-identification system detecting a base-call-error pattern from grouped base-call-error rates in accordance with one or more embodiments of the present disclosure.

FIG. 5 illustrates the variation-source-identification system identifying a sample base-call-error pattern for one or more sample sequencing runs in accordance with one or more embodiments of the present disclosure.

FIGS. 6A-6C illustrate the variation-source-identification system determining contribution metrics indicating contributions of sequencing-pipeline materials to base-call errors from the sequencing pipeline in accordance with one or more embodiments of the present disclosure.

FIGS. 7A-7C illustrate a series of example variance components analysis outputs generated by the variation-source-identification system as part of identifying failure sources contributing to base-call errors in accordance with one or more embodiments of the present disclosure.

FIG. 8 illustrates example percent assignable cause variations for sequencing pipeline materials contributing to variations in insertion and deletion (INDEL) lengths in accordance with one or more embodiments of the present disclosure.

FIGS. 9A-9B illustrate an example series of graphical user interfaces including a notification graphical user interface from the variation-source-identification system including a failure mode notification and an error-pattern-analysis graphical user interface in accordance with one or more embodiments of the present disclosure.

FIG. 10 illustrates a series of acts for detecting a base-call-error pattern from the sequencing data of a sequencing pipeline and determining a failure source for a base-call-error type based on the base-call-error pattern in accordance with one or more embodiments of the present disclosure.

FIG. 11 illustrates a block diagram of an example computing device in accordance with one or more embodiments of the present disclosure.

### DETAILED DESCRIPTION

This disclosure describes one or more embodiments of a variation-source-identification system that identifies a base-call-error pattern from the sequencing data of a sequencing pipeline and determines a failure source based on the base-call-error pattern. In one or more embodiments, the variation-source-identification system generates base calls for a reference genome to determine base-call-error rates for individual bases. The variation-source-identification system can further identify a base-call-error pattern based on the base-call-error rates. As a point of comparison, the variation-source-identification system further identifies a sample base-call-error pattern that corresponds to the base-call-error pattern. Based on the correlation between the base-call-error pattern and the sample base-call-error pattern, the variation-source-identification system can determine a failure source (e.g., based on percent assignable cause variations) for variations within sequencing data for the sequencing pipeline.

To illustrate, in one or more embodiments, the variation-source-identification system determines base-call-error rates at which nucleotide-base calls generated by a sequencing pipeline differ from reference bases in a reference genome. The variation-source-identification system can detect a base-call-error pattern from the base-call-error rates grouped according to base-call-error types. In some embodiments, the variation-source-identification system identifies a sample base-call-error pattern for one or more sample sequencing runs that utilize one or more sequencing pipelines corresponding to the sequencing pipeline based on the base-call-error pattern. The variation-source-identification system can further determine a failure source for a base-call-error type corresponding to the sequencing pipeline based on a correlation between the base-call-error pattern and the sample base-call-error pattern.

As mentioned, the variation-source-identification system can determine base-call-error rates at which nucleotide-base calls differ from reference bases. In particular, the variation-source-identification system can utilize a reference genome having a known sequence of reference bases. In some embodiments, the variation-source-identification system utilizes a confusion matrix to indicate correct and incorrect base calls of the sequencing run. Additionally, in one or more embodiments, the variation-source-identification system further normalizes data from the confusion matrix. In any case, the variation-source-identification system can utilize a reference genome to accurately identify correct and incorrect base calls generated by a sequencing pipeline.

The variation-source-identification system can further detect a base-call-error pattern from the base-call-error rates grouped according to base-call-error types. In particular, the variation-source-identification system can identify base-call-error types indicating a correct base call and an incorrect base call. For example, the variation-source-identification system can determine the number of times when a correct guanine (G) base call is erroneously identified as an incorrect adenosine (A) base call. Additionally, in some embodiments, the variation-source-identification system can generate more detailed base-call-error patterns by grouping incorrect base calls based on different neighboring nucleotide bases. For instance, the variation-source-identification system can determine when a G base call is incorrectly called as an A when flanked by A nucleotides on both sides as opposed to an A and a cytosine (C). Generally, the variation-source-identification system can generate a base-call-error pattern comprising the groups of base-call-error types and different neighboring nucleotide bases.

Based on the base-call-error pattern from the sequencing data of a sequencing pipeline, the variation-source-identification system can further identify a sample base-call-error pattern for one or more sample sequencing runs that utilize one or more sequencing pipelines corresponding to the sequencing pipeline. Generally, the variation-source-identification system utilizes a statistical model, such as Variance Components Analysis (VCA), to analyze sample sequencing runs and manufacturing data to estimate the variability of various factors. In one example, the variation-source-identification system can define sets of sample sequencing runs that utilize similar manufacturing materials based on manufacturing identification data. The variation-source-identification system detects sample base-call-error patterns for the sets of sample sequencing runs and utilizes a statistical model to determine assignable cause variations for sequencing pipeline materials, chemistry, or software contributing to the sample base-call errors.

Based on a correlation between the base-call-error pattern from data of a sequencing pipeline and the sample base-call-error pattern from sample sequencing runs, the variation-source-identification system can further determine a failure source for a base-call-error type. As mentioned, in some cases, the variation-source-identification system utilizes a statistical model to estimate the effects of hardware, chemistry, and software on sequencing run data. By identifying sample base-call-error patterns that correspond with the base-call-error pattern, the variation-source-identification system can determine the failure source for the base-call-error type.

Having identified a failure source, in one or more embodiments, the variation-source-identification system provides, for display on a computing device associated with the sequencing pipeline, a notification indicating the failure source. For instance, the variation-source-identification system can provide a notification that indicates one or more failure sources that negatively impact a sequencing run. The variation-source-identification system may also provide, via the notification, a breakdown of potential failure sources and probabilities that the potential failure sources are negatively affecting the sequencing run.

The variation-source-identification system provides several technical benefits relative to existing sequencing systems. In particular, the variation-source-identification system can improve the accuracy of detecting systemic error sources relative to existing sequencing systems.

More specifically, the variation-source-identification system utilizes base-call-error rates for a reference genome to infer specific failure sources that negatively impact sequencing runs. In contrast to existing systems that rely on a Phred algorithm to determine quality scores that estimate a likelihood that an individual base call is incorrect, the variation-source-identification system can accurately identify systemic error sources that originate in various parts along a sequencing pipeline. For instance, the variation-source-identification system can identify failure sources in machinery, reagents, chemistry, or software. Additionally, in contrast to conventional error correction tools that introduce new errors into a nucleotide sequence, the variation-source-identification system analyzes base-call data without negatively impacting read length or coverage bias.

The variation-source-identification system can also improve the efficiency of detecting sequencing failure sources relative to existing sequencing systems. By utilizing sequencing base-call data to efficiently identify failure sources, the variation-source-identification system obviates the need to run and re-run multiple sequencing cycles to achieve high quality data and thereby more efficiently uses chemical reagents than existing sequencing systems. In some embodiments, the variation-source-identification system can also improve efficiency by providing a notification of potential failure sources in real time (e.g., a graphical indication of an error code). For example, while many existing systems rely on algorithms, such as Louvian community detection algorithms to generate similarity scores between individual read pairs within a given segment, the variation-source-identification system can review the base-call data of an entire nucleotide sequence to accurately identify failure sources. Thus, unlike many existing systems that require excessive computational resources to identify and correct sequencing errors, the variation-source-identification system can provide an efficient interface for identifying and correcting potential failure sources.

By providing timely notifications of failure sources, the variation-source-identification system can accordingly reduce the amount of wasted reagents on sequencing runs with identified errors and trouble shoot (and correct) failure sources within a sequencing pipeline. With an identified failure source for a base-call-error pattern, the variation-source-identification system can target raw materials and processes to fix or improve raw materials produced in the future. Similarly, the variation-source-identification system can end a sequencing cycle or sequencing run early to correct identified failure sources and thereby preserve reagents of a current cycle or run. Once a failure source has been remedied for a sequencing pipeline, a sequencing system that uses the remedied sequencing pipeline to determine sequences of sample genomes (or other nucleic-acid polymers) can improve the base-call-error rates over previous sequencing runs. By identifying new base-call-error patterns in both manufacturing and field data, the variation-source-identification system can also improve base-call-error rates and the accuracy of predicted failure sources in future sequencing runs.

In addition to improved accuracy and efficiency, the variation-source-identification system improves flexibility relative to existing sequencing systems. Unlike conventional in-machine sensors, in some embodiments, the variation-source-identification system is platform agnostic and does not require the use of additional hardware. In particular, the variation-source-identification system flexibly utilizes base-call-error rates for a sequenced reference genome that is readily accessible to numerous sequencing platforms. Furthermore, the variation-source-identification system is not limited to a single reference genome, rather, the variation-source-identification system can flexibly utilize sequencing from any known reference genome to generate base-call-error patterns for sequencing runs. Thus, the variation-source-identification system can be implemented and utilized by existing sequencing systems without the requirement for additional hardware.

As illustrated by the foregoing discussion, the present disclosure utilizes a variety of terms to describe features and advantages of the variation-source-identification system. Additional detail is now provided regarding the meaning of such terms. For example, as used herein, the term “base-call-error rate” refers to an indication of a fraction, frequency, percentage, or other portion at which incorrect nucleotide-base calls are determined. In particular, base-call-error rate can indicate a fraction, frequency, or percentage at which nucleotide-base calls generated by a sequencing pipeline differ from reference bases in a reference genome. In one example, a base-call-error rate comprises a count of instances where the sequencing pipeline generated an incorrect nucleotide-base call (e.g., erroneously called an adenine base call for a guanine base).

As used herein, the term “nucleotide-base call” (or simply “base call”) refers to a determination or prediction of a particular nucleotide base (or nucleotide-base pair) for a genomic coordinate of a sample genome or for an oligonucleotide during a sequencing cycle. In particular, a nucleotide-base call can indicate (i) a determination or prediction of the type of nucleotide base that has been incorporated within an oligonucleotide on a nucleotide-sample slide (e.g., read-based nucleotide-base calls) or (ii) a determination or prediction of the type of nucleotide base that is present at a genomic coordinate or region within a genome, including a variant call or a non-variant call in a digital output file. In some cases, for a nucleotide-fragment read, a nucleotide-base call includes a determination or a prediction of a nucleotide base based on intensity values resulting from fluorescent-tagged nucleotides added to an oligonucleotide of a nucleotide-sample slide (e.g., in a well of a flow cell). Alternatively, a nucleotide-base call includes a determination or a prediction of a nucleotide base from chromatogram peaks or electrical current changes resulting from nucleotides passing through a nanopore of a nucleotide-sample slide. By contrast, a nucleotide-base call can also include a final prediction of a nucleotide base at a genomic coordinate of a sample genome for a variant call file or other base-call-output file—based on nucleotide-fragment reads corresponding to the genomic coordinate. Accordingly, a nucleotide-base call can include a base call corresponding to a genomic coordinate and a reference genome, such as an indication of a variant or a non-variant at a particular location corresponding to the reference genome. Indeed, a nucleotide-base call can refer to a variant call, including but not limited to, a single nucleotide polymorphism (SNP), an insertion or a deletion (indel), or base call that is part of a structural variant. As suggested above, a single nucleotide-base call can be an adenine (A) call, a cytosine (C) call, a guanine (G) call, or a thymine (T) call.

As used herein, the term “failure source” refers to a cause of a given base-call error, base-call-error rate, or base-call-error type. In particular, a failure source refers to a specific issue found at various components within a sequencing pipeline that negatively impact nucleotide-base calling. For instance, failure sources can include issues or problems impacting hardware, chemistry, or software that cause errors, such as miscalled nucleotide bases. Examples of failure sources found in hardware can include faulty parts of a sequencing machine and degraded or otherwise faulty consumable products. Examples of failure sources found in chemistry can include consumable products that are negatively impacted when they interact with other consumable products, the environment, or parts of a sequencing machine. Failure sources found in software can comprise computing errors or other irregularities stemming from the computing processes utilized within a sequencing pipeline.

As used herein, the term “reference genome” refers to a digital nucleic-acid sequence assembled as a representative example (or representative examples) of genes for an organism. Regardless of the sequence length, in some cases, a reference genome represents an example set of genes or a set of nucleic-acid sequences in a digital nucleic-acid sequenced determined by scientists or statistical models as representative of an organism of a particular species. For example, a reference genome can comprise a PhiX genome. As a further example, a linear human reference genome may be GRCh3 8 or other versions of reference genomes from the Genome Reference Consortium. A reference genome is composed of a known sequence of reference bases. As used herein, the term “reference bases” refers to nucleotide bases that compose a reference genome. In particular, a sequence of reference bases can be used as a control for sequencing runs.

As used herein, the term “sequencing pipeline” refers to various physical elements and software used to determine a sequence of a nucleic-acid polymer or whole genome. In particular, a sequencing pipeline can include a nucleic-acid-sequence-extraction method and corresponding reagents and corresponding equipment for extraction; a sequencing device and corresponding reagents, equipment, and/or reactions utilized in a sequencing run; and a sequence-analysis software. For example, a sequencing pipeline can include a particular model of sequencing device and the corresponding reagents that the sequencing device utilizes within a series of events to generate a nucleotide-base sequence.

As used herein, the term “similar manufacturing materials” refers to materials utilized within one or more sequencing pipelines with shared characteristics. In particular, similar manufacturing materials can include two materials of the same type or same or overlapping crate or manufacturing identifier that also have shared characteristics. As explained below, in some cases, the variation-source-identification system truncates manufacturing identification data for sequencing devices, sequencing-device parts, consumable products, nucleotide-sample slides, and other materials to identify similar manufacturing materials. Accordingly, similar manufacturing materials can include sequencing device parts, consumable products, nucleotide-sample slides, and other materials that are the same or similar in composition or build. In some embodiments, similar manufacturing materials can include two reagents of the same type that are created using the same raw materials, through the same process, and at the same time.

As used herein, the term “base-call-error pattern” refers to a distinctive or unique combination of base-call errors. In particular, a base-call-error pattern can include a signature or distinctive series of various base-call errors across one or more sequencing runs. For example, a base-call-error pattern can refer to a signature indicating the volume of base-call errors of each base-call-error types across one or more sequencing runs. Additionally, the base-call-error pattern can include a pattern indicating the volume of base-call errors of particular types (e.g., incorrectly calling an A instead of a T) organized according to different neighboring nucleotide bases.

As further used herein, the term “sample sequencing run” refers to a nucleotide sequencing run with known variables from a sequencing pipeline. In particular, a sample sequencing run generates sample sequencing data by utilizing known manufacturing data for one or more sequencing pipelines. In some embodiments, a sample sequencing run comprises test sequencing runs that utilize manufacturing materials with known manufacturing identification data. For example, sample sequencing runs can comprise quality test runs conducted using nucleic-acid-sequence-extraction methods, sequencing devices, or sequence-analysis software to ensure that the nucleic-acid-sequence-extraction methods, sequencing devices, or sequence-analysis software pass corresponding quality standards.

Similarly, as used herein, the term “sample base-call-error pattern” refers to a distinctive or unique combination of base-call errors present within one or more sample sequencing runs. In particular, a sample base-call-error pattern can refer to a signature or distinctive series of base-call errors made by a sequencing pipeline during a sample sequencing run. In one example, sample base-call-error patterns indicate volumes of various base-call errors when the sequencing device or sequence-analysis software is analyzing sample data.

As used herein, the term “base-call-error type” refers to a category of base-call error. In particular, a base-call-error type indicates a specific erroneous base call determined instead of a correct base call. For example, a base-call-error type can include an A base (e.g., here, the correct base call is A) was miscalled by a sequencing system as a G. By contrast, a different base-call-error type can include an A base was miscalled by a sequencing system as a T. In one example, base-call-error types are determined by comparing a known sequence of reference bases with nucleotide-base calls.

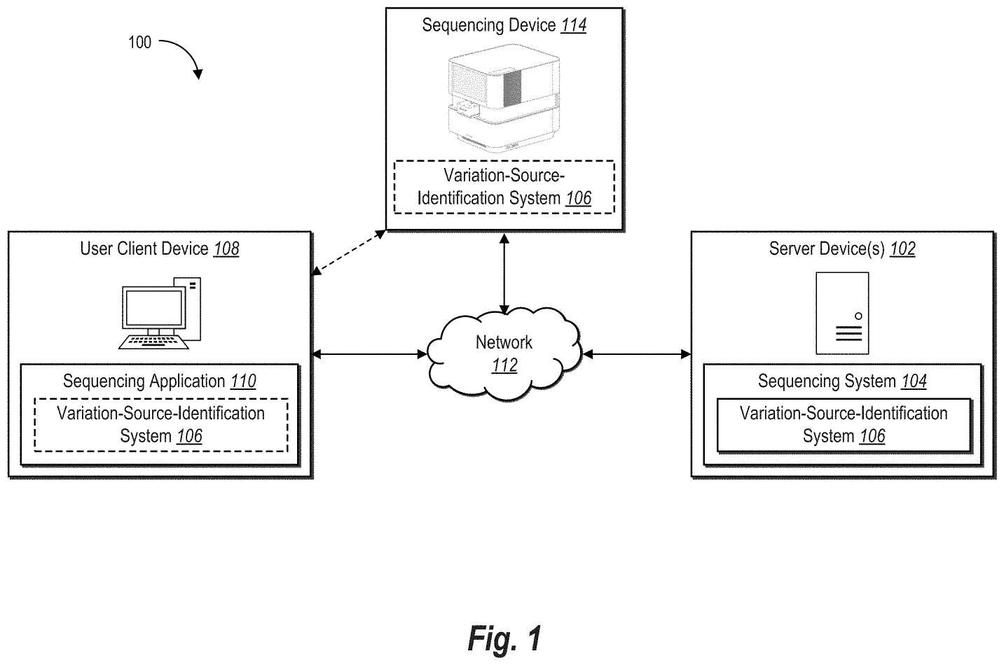

Additional detail will now be provided regarding a variation-source-identification system in relation to illustrative figures portraying example embodiments and implementations of the variation-source-identification system. For example, FIG. 1 illustrates a schematic diagram of a system environment (or “environment”)100 in which a variation-source-identification system 106 operates in accordance with one or more embodiments. As illustrated, the environment 100 includes one or more server device(s) 102 connected to a user client device 108 and a sequencing device 114 via a network 112. While FIG. 1 shows an embodiment of the variation-source-identification system 106, alternative embodiments and configurations are possible.

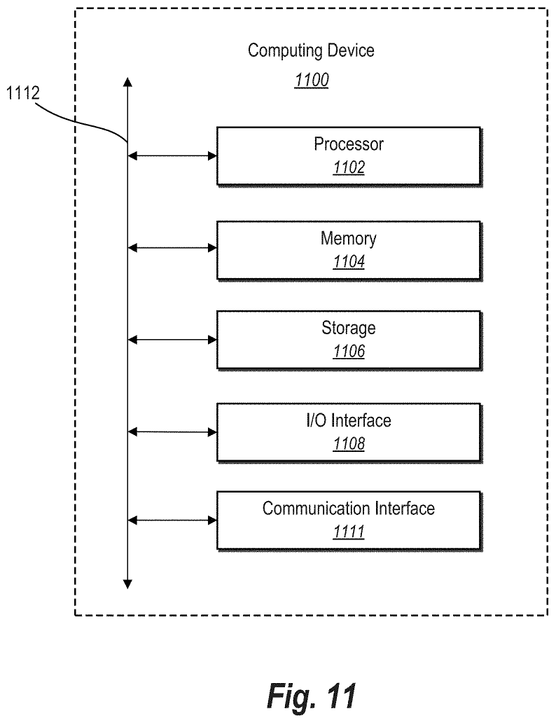

As further shown in FIG. 1, the server device(s) 102, the user client device 108, and the sequencing device 114 are connected via the network 112. Each of the components of the environment 100 can communicate via the network 112. The network 112 comprises any suitable network over which computing devices can communicate. Example networks are discussed in additional detail below in relation to FIG. 11.

As shown in FIG. 1, the environment 100 includes the sequencing device 114. The sequencing device 114 comprises a device for sequencing a nucleic-acid polymer or a whole genome. In some embodiments, the sequencing device 114 analyzes samples to generate data utilizing computer implemented methods and systems described herein either directly or indirectly on the sequencing device 114. In one or more embodiments, the sequencing device 114 utilizes Sequencing By Synthesis (SBS) to sequence nucleic-acid polymers. As shown, in some embodiments, the sequencing device 114 bypasses the network 112 and communicates directly with the user client device 108.

As further depicted by FIG. 1, the environment 100 includes the server device(s) 102. The server device(s) 102 may generate, receive, analyze, store, receive, and transmit electronic data, such as data for sequencing nucleic-acid polymers. The server device(s) 102 may receive data from the sequencing device 114. For example, the server device(s) 102 may gather and/or receive sequencing data including nucleotide-base call data, quality data, and other data relevant to sequencing nucleic-acid polymers. The server device(s) 102 may also communicate with the user client device 108. In particular, the server device(s) 102 can send nucleic-acid polymer sequences, error data, and other information to the user client device 108. In some embodiments, the server device(s) 102 comprise a distributed server where the server device(s) 102 include a number of server devices distributed across the network 112 and located in different physical locations. The server device(s) 102 can comprise a content server, an application server, a communication server, a web-hosting server, or another type of server.

As further shown in FIG. 1, the server device(s) 102 can include the sequencing system 104. Generally, the sequencing system 104 analyzes sequencing data received from the sequencing device 114 to determine nucleotide sequences for nucleic-acid polymers. For example, the sequencing system 104 can receive raw data (e.g., base-call data for nucleotide-fragment reads) from the sequencing device 114 and determine a nucleic acid sequence for a sample. To illustrate, the sequencing system 104 can receive nucleotide-fragment reads from the sequencing device 114, and the sequencing system 104 generates nucleotide-base calls for a genome from the nucleotide-fragment reads. In some embodiments, the sequencing system 104 determines the sequences of nucleobases in DNA and/or RNA. In addition to processing and determining sequences for nucleic-acid polymers, the sequencing system 104 also analyzes sequencing data to detect irregularities in individual or multiple sequencing cycles. For instance, the sequencing system 104 can detect base-call errors within a sequencing run by comparing nucleotide-base calls for a reference genome against known reference bases for the reference genome.

As illustrated in FIG. 1, the sequencing system 104 includes the variation-source-identification system 106. Generally, the variation-source-identification system 106 analyzes data from the sequencing device 114 to determine a failure source for a sequencing run associated with the sequencing device 114. More specifically, in some embodiments, the variation-source-identification system 106 determines base-call-error rates at which nucleotide-base calls generated by a sequencing pipeline differ from reference bases in a reference genome. The variation-source-identification system 106 can further detect a base-call-error pattern from the base-call-error rates grouped according to base-call-error types. Based on the base-call-error patterns, the variation-source-identification system 106 can identify a sample base-call-error pattern for one or more sample sequencing runs that utilize one or more sequencing pipelines corresponding to the sequencing pipeline. Based on the correlation between the base-call-error pattern and the sample base-call-error pattern, the variation-source-identification system 106 can determine a failure source for a base-call-error type corresponding to the sequencing pipeline.

The environment 100 illustrated in FIG. 1 further includes the user client device 108. The user client device 108 can generate, store, receive, and send digital data. In particular, the user client device 108 can receive sequencing data from the sequencing device 114. Furthermore, the user client device 108 may communicate with the server device(s) 102 to receive nucleotide-base calls, nucleotide sequences, and reports of irregularities within a sequencing run such as notifications indicating potential failure sources for errors in nucleotide-base calls. The user client device 108 can present sequencing data and notifications of failure sources to a user associated with the user client device 108.

The user client device 108 illustrated in FIG. 1 may comprise various types of client devices. For example, in some embodiments, the user client device 108 includes non-mobile devices, such as desktop computers or servers, or other types of client devices. In yet other embodiments, the user client device 108 includes mobile devices, such as laptops, tablets, mobile telephones, smartphones, etc. Additional details with regard to the user client device 108 are discussed below with respect to FIG. 11.

As further illustrated in FIG. 1, the user client device 108 includes a sequencing application 110. The sequencing application 110 may be a web application or a native application on the user client device 108 (e.g., a mobile application, desktop application, etc.). The sequencing application 110 can comprise instructions that (when executed) cause the user client device 108 to receive data from the variation-source-identification system 106 and present sequencing data. Furthermore, the sequencing application 110 can comprise instructions that (when executed) cause the user client device 108 to provide a notification indicating potential failure sources affecting a sequencing run.

As further illustrated in FIG. 1, the variation-source-identification system 106 may be located on the user client device 108 as part of the sequencing application 110. As illustrated, in some embodiments, the variation-source-identification system 106 is implemented by (e.g., located entirely or in part) on the user client device 108. In yet other embodiments, the variation-source-identification system 106 is implemented by one or more other components of the environment 100. In particular, the variation-source-identification system 106 can be implemented in a variety of different ways across the server device(s) 102, the user client device 108, and the sequencing device 114.

Though FIG. 1 illustrates the components of environment 100 communicating via the network 112, in some embodiments, the components of environment 100 communicate directly with each other, bypassing the network. For instance, and as previously mentioned, the user client device 108 can communicate directly with the sequencing device 114. Additionally, the user client device 108 can communicate directly with the variation-source-identification system 106, bypassing the network 112. Moreover, the variation-source-identification system 106 can access one or more databases housed on the server device(s) 102 or elsewhere in the environment 100.

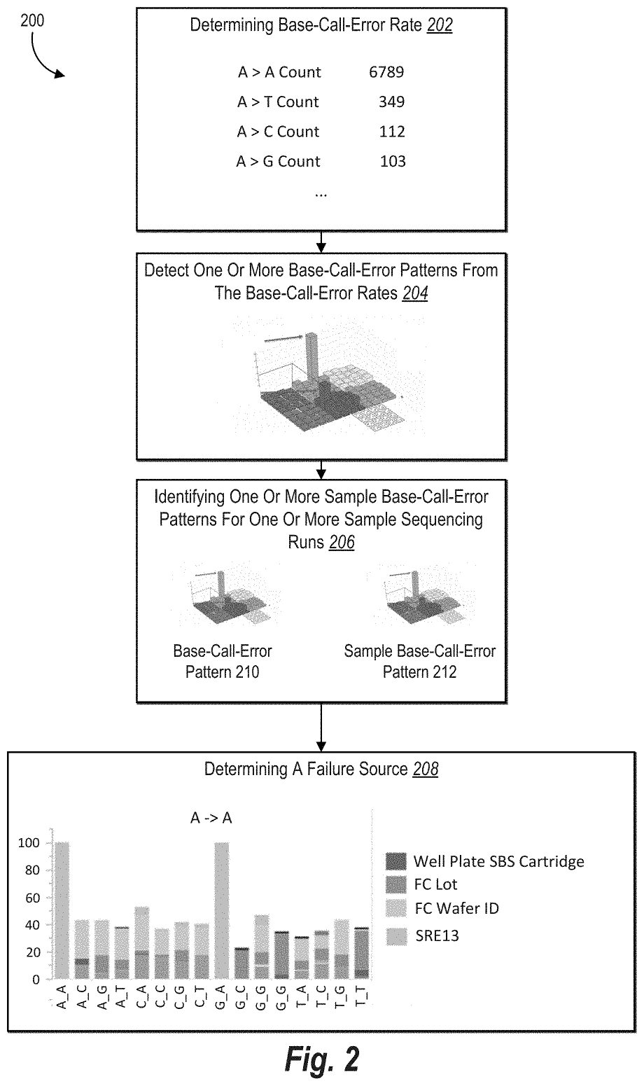

As previously mentioned, the variation-source-identification system 106 can determine a failure source for a base-call-error type corresponding to a sequencing pipeline. The following figures and paragraphs provide additional detail regarding how the variation-source-identification system 106 determines one or more failure sources in accordance with some embodiments. FIG. 2 and the corresponding paragraph provide a general overview of acts that the variation-source-identification system 106 performs as part of determining a failure source in accordance with one or more embodiments. As shown in FIG. 2, the variation-source-identification system 106 determines incorrect base-calls and a base-call-error pattern based on the combined incorrect base-calls. The variation-source-identification system 106 further compares the base-call-error pattern with sample base-call-error patterns to identify a corresponding sample base-call-error pattern. Based on the corresponding sample base-call-error pattern, the variation-source-identification system 106 can determine a failure source.

As illustrated in FIG. 2, the series of acts 200 includes an act 202 of determining a base-call-error rate. In particular, the variation-source-identification system 106 determines base-call-error rates at which nucleotide-base calls generated by a sequencing pipeline differ from reference bases in a reference genome. Generally, the variation-source-identification system 106 determines error rates at which nucleotide-base calls generated by the sequencing pipeline differ from the known reference bases of the reference genome. In some embodiments, the variation-source-identification system 106 compares the nucleotide-base calls for the reference genome (as determined by a sequencing pipeline from nucleotide-fragment reads) with the reference bases of the reference genome. Based on a comparison of the nucleotide-base calls and the reference bases, the variation-source-identification system 106 identifies both incorrect nucleotide-base calls and correct nucleotide-base calls generated by the sequencing pipeline. For example, and as illustrated in FIG. 2, the variation-source-identification system 106 can determine instances when a sequencing system erroneously generates an incorrect nucleotide-base call of T in place of a correct nucleotide-base call of A representing a reference base.

The variation-source-identification system 106 further determines error rates for incorrect base calls. In some embodiments, the variation-source-identification system 106 determines the number of instances that a sequencing system in a sequencing pipeline generates an incorrect nucleotide-base call. For instance, and as illustrated in FIG. 2, the variation-source-identification system 106 determines that the sequencing pipeline correctly predicted an A nucleotide-base call in 6798 instances. In contrast, the sequencing pipeline incorrectly called A bases incorrectly as T in 349 instances, C in 112 instances, and Gin 103 instances. As suggested above, in some embodiments, the variation-source-identification system 106 further determines a normalized base-call-error rate to standardize the base-call-error rate.

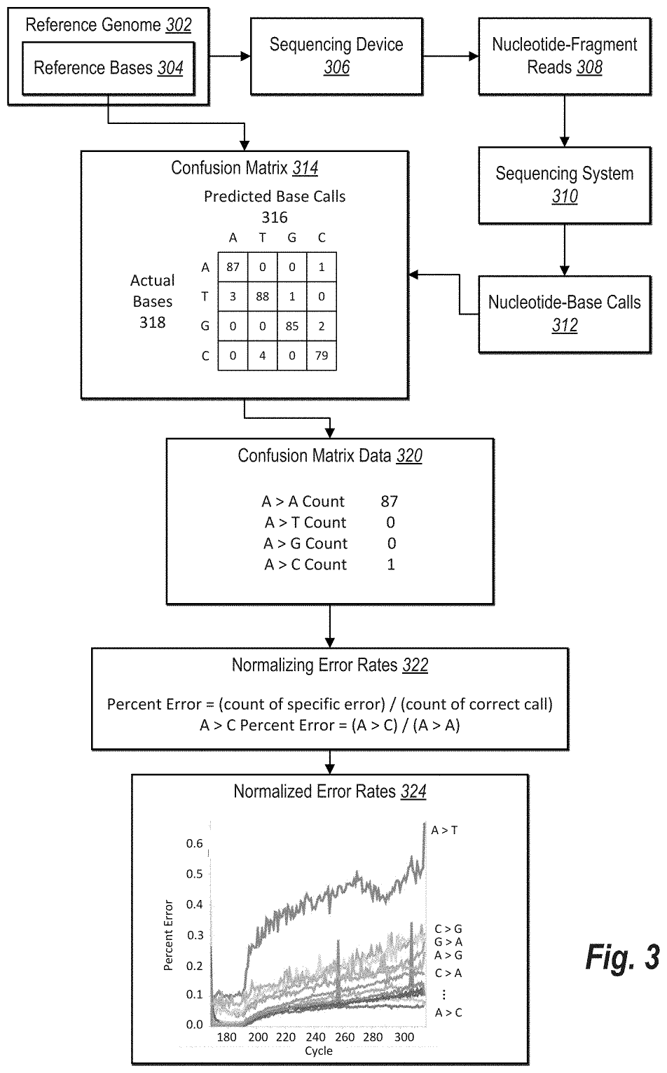

Though FIG. 2 illustrates incorrect nucleotide-base calls for A bases, the variation-source-identification system 106 determines base-call error rates for all bases within a nucleotide sequence. FIG. 3 and the corresponding paragraph provide additional detail regarding determining base-call-error rates in accordance with one or more embodiments.

As further illustrated in FIG. 2, the variation-source-identification system 106 performs an act 204 of detecting one or more base-call-error patterns from the base-call-error rates. Generally, the variation-source-identification system 106 groups base-call-error rates and determines the base-call-error patterns based on the grouped base-call-error rates. In some embodiments, for instance, the variation-source-identification system 106 simply groups the base-call-error patterns according to base-call-error types. For example, the variation-source-identification system 106 can designate an incorrect nucleotide-base call T in place of an A (e.g., A→T) as a single base-call-error type. Additionally, or alternatively, the variation-source-identification system 106 groups base-call-error rates by different neighboring nucleotide bases. To illustrate, the variation-source-identification system 106 can, for the base-call-error type A→T, further distinguish groupings based on the neighboring nucleotide bases. For instance, an A→T base-call-error type can be flanked by an A and an A (i.e., A_A).

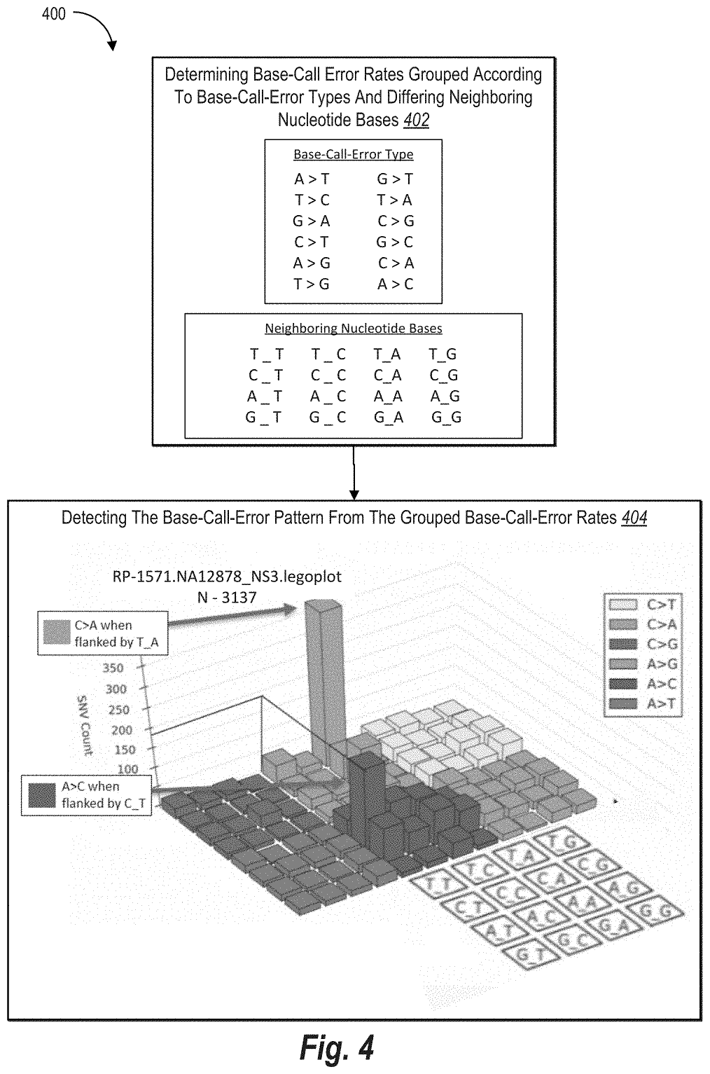

FIG. 2 illustrates a 3-dimensional chart representing a base-call-error pattern for a sequencing pipeline. The 3-dimensional chart represents base-call-error rates grouped by both base-call-error type and neighboring nucleotide bases. As described further below, FIG. 4 and the corresponding discussion provide additional detail relating to detecting base-call-error patterns in accordance with one or more embodiments.

FIG. 2 also illustrates the variation-source-identification system 106 performing an act 206 of identifying one or more sample base-call-error patterns for one or more sample sequencing runs. Generally, the variation-source-identification system 106 identifies sample-base-call-error patterns that fall within a threshold similarity with the base-call-error pattern. In particular, the variation-source-identification system 106 generates sample base-call-error patterns using sample sequencing runs. The variation-source-identification system 106 further utilizes a statistical method and manufacturing data associated with the sample sequencing runs to determine failure sources of variation within the sequencing runs. For example, and as illustrated in FIG. 2, the variation-source-identification system 106 determines that sample base-call-error pattern 212 is within a threshold similarity of base-call-error pattern 210.

As part of the series of acts 200 illustrated in FIG. 2, the variation-source-identification system 106 performs an act 208 of determining a failure source. Based on a correlation between the base-call-error pattern and the sample base-call-error pattern, the variation-source-identification system 106 determines a failure source for the base-call-error type corresponding to the sequencing pipeline. In some embodiments, the variation-source-identification system 106 utilizes a statistical model to determine contribution metrics indicating probabilities of sequencing-pipeline materials contributing to base-call errors from the sequencing pipeline. The variation-source-identification system 106 can further determine the failure source for the base-call-error types based on the contribution metrics.

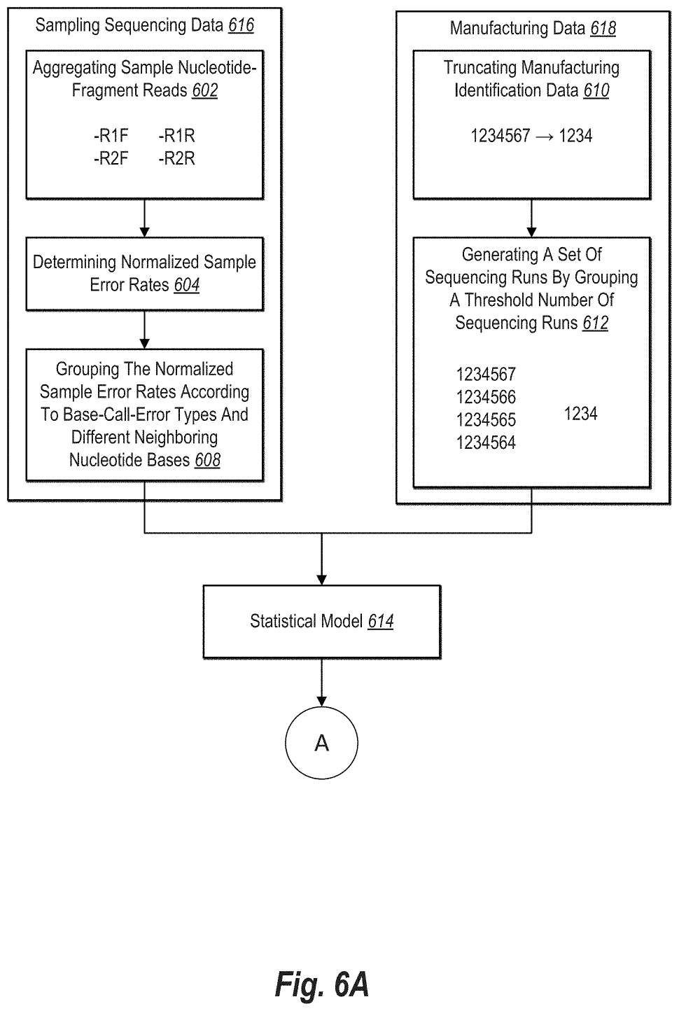

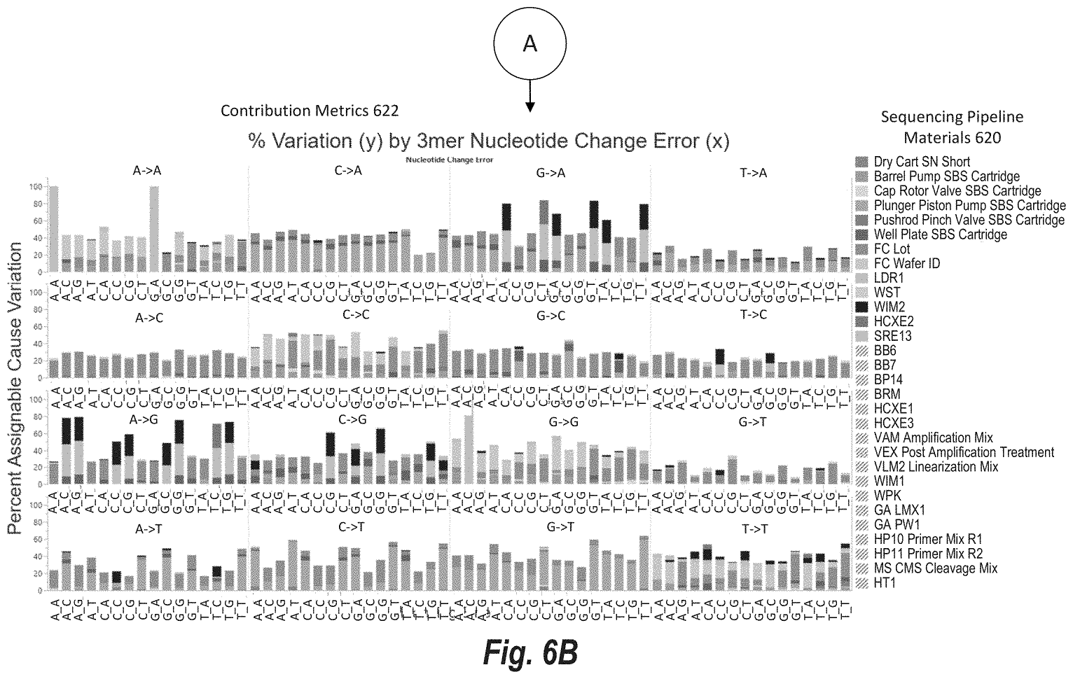

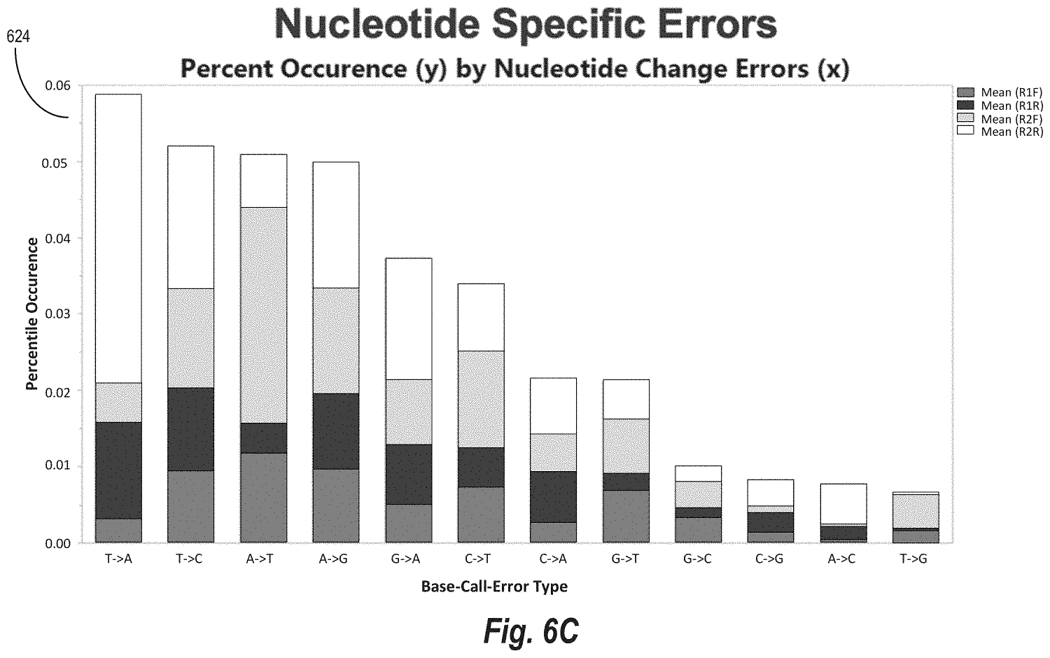

As an example of such a statistical model, in some embodiments, the variation-source-identification system 106 utilizes a variance components model to determine assignable cause variations for sequencing-pipeline materials contributing to base-call errors attributable to the sequencing pipeline. FIGS. 6A-6C and the corresponding paragraphs provide additional detail regarding the variation-source-identification system 106 determining a failure source for the base-call-error type corresponding to the sequencing pipeline.

FIG. 2 provides a general overview of acts the variation-source-identification system 106 performs to determine one or more failure sources corresponding to a sequencing pipeline. The following figures and paragraphs provide additional details regarding acts within the series of acts illustrated in FIG. 2. For example, FIG. 3 and the corresponding paragraphs provide additional detail relating to the variation-source-identification system 106 determining base-call-error rates in accordance with one or more embodiments.

As illustrated in FIG. 3, the variation-source-identification system 106 utilizes a sequencing device 306 to generate nucleotide-fragment reads 308 for a reference genome 302. The variation-source-identification system 106 further utilizes a sequencing system 310 (e.g., the sequencing system 104) to generate nucleotide-base calls 312 based on the nucleotide-fragment reads 308. The variation-source-identification system 106 generates and utilizes a confusion matrix 314 to compare the nucleotide-base calls 312 with reference bases 304 of the reference genome 302. The variation-source-identification system 106 further processes confusion matrix data 320 output by the confusion matrix 314 by performing an act 322 of normalizing error rates to generate normalized error rates 324.

As further illustrated in FIG. 3, the variation-source-identification system 106 utilizes the reference genome 302 comprising the reference bases 304 to generate the nucleotide-base calls 312. Generally, the reference genome 302 contains a known sequence of the reference bases 304. The variation-source-identification system 106 utilizes the reference genome 302 as a control by which to measure accuracy of nucleotide-base calls. In some embodiments, for instance, the reference genome 302 comprises a PhiX genome. PhiX is an icosahedral, nontailed bacteriophage with a single-stranded DNA. In some embodiments, the variation-source-identification system 106 utilizes other control genomes as the reference genome 302. For instance, the reference genome 302 can comprise a spike-in genomic DNA or a mutated sequence that exhibits or simulates mutagenesis.

As further illustrated in FIG. 3, the variation-source-identification system 106 utilizes the sequencing device 306 and the sequencing system 310 to generate the nucleotide-base calls 312 for the reference genome 302. Generally, the sequencing device 306 generates the nucleotide-fragment reads 308 that indicate sequences of various fragments from within the reference genome 302. The sequencing system 310 aligns the nucleotide-fragment reads 308 with the reference genome 302 to generate the nucleotide-base calls 312. Because the nucleotide-fragment reads 308 may include incorrect nucleotide-base calls, the nucleotide-fragment reads 308 may not align well with the reference genome 302. For instance, a number of nucleotide-base calls from the nucleotide-fragment reads 308 may not match the reference genome 302 and result in a mapping-quality metrics below a threshold metric (e.g., below a relative MAPQ score or below a MAPQ 40). Similarly, because the sequencing device 306 or other parts of a sequencing pipeline include faulty parts, reagents, or software, the sequencing system 104 may generate incorrect nucleotide-base calls as part of the nucleotide-base calls 312.

As further illustrated in FIG. 3, the variation-source-identification system 106 utilizes the confusion matrix 314 to detect errors within the nucleotide-base calls 312. Generally, the confusion matrix 314 evaluates the performance of the sequencing device 306 and the sequencing system 310. In some embodiments, the confusion matrix 314 comprises a table as illustrated in FIG. 3. The table includes different classes for predicted base calls 316 and actual bases 318. The predicted base calls 316 represent base calls from the nucleotide-base calls 312. The actual bases 318 represent the reference bases 304, which are known.

The variation-source-identification system 106 utilizes the confusion matrix 314 by generating counts for each instance where the sequencing pipeline correctly predicted a nucleotide-base call. The variation-source-identification system 106 also utilizes the confusion matrix 314 to provide details regarding incorrect nucleotide-base calls. For example, the variation-source-identification system 106 can utilize the confusion matrix 314 to indicate the actual base and the incorrect nucleotide-base call. For instance, the variation-source-identification system 106 determines, utilizing the confusion matrix 314, a single instance where the sequencing pipeline determine an incorrect C base call for an actual A base.

As suggested above, the variation-source-identification system 106 utilizes the confusion matrix 314 to generate the confusion matrix data 320. The confusion matrix data 320 indicates the number of instances where the sequencing pipeline generated correct and incorrect nucleotide-base calls. The numbers in the confusion matrix 314 indicate the number of instances that the sequencing system 310 generated correct or incorrect nucleotide-base calls.

For example, the confusion matrix 314 indicates that the sequencing system 310 correctly identified A bases in 87 instances, T bases in 88 instances, G bases in 85 instances, and C bases in 79 instances. By contrast, the variation-source-identification system 106 utilizes the confusion matrix 314 to determine that for the actual base T, the sequencing system 310 generated the incorrect A base-call in three instances. Similarly, the variation-source-identification system 106 identifies one A→C call, one T→G call, two G→C calls, and four C→T calls. The confusion matrix data 320 illustrated in FIG. 3 includes confusion matrix data specifically for actual A bases.

In some embodiments, and as illustrated in FIG. 3, the variation-source-identification system 106 performs the act 322 of normalizing error rates. By performing the act 322, the variation-source-identification system 106 can accurately compare the results of one sequencing run with another sequencing run regardless of the number of nucleotide-base calls. The variation-source-identification system 106 may utilize different normalization methods to perform the act 322. For example, in some embodiments, the variation-source-identification system 106 performs the act 322 by dividing the number of instances of a specific error with the number of instances of the corresponding correct nucleotide-base call.

To illustrate such normalization, the variation-source-identification system 106 illustrated in FIG. 3 calculates a normalized percent error by dividing the instances of A→C errors by the number of instances of correct A→A calls. In this example, the variation-source-identification system 106 divides 1 (A→C errors) by 87 (A→A correct calls). In other embodiments, the variation-source-identification system 106 utilizes different normalization methods, such as scaling to range, log scaling, and other methods to perform the act 322 of normalizing error rates.

FIG. 3 further illustrates the normalized error rates 324. The variation-source-identification system 106 normalizes each specific error according to the methods described above. Generally, and as illustrated in FIG. 3, error rates within sequencing cycles tend to be nucleotide specific. The variation-source-identification system 106 takes the nucleotide-specificity of error rates into account by determining normalized error rates based on actual and incorrect nucleotide bases. For example, as illustrated in FIG. 3, A→T errors are a larger contributor to the general error rate than other base-call-error types.

Additionally, in some embodiments, the variation-source-identification system 106 normalizes error rates for each sequencing cycle. The graph illustrated in FIG. 3 displays normalized error rates for each base-call-error type across sequencing cycles. For example, the variation-source-identification system 106 determines that the A→T base-call-error type dramatically increases between sequencing cycles 150 and 200.

FIG. 3 and the corresponding paragraphs describe the variation-source-identification system 106 determining base-call-error rates by generating normalized error rates in accordance with one or more embodiments. As previously mentioned, the variation-source-identification system 106 may further detect a base-call-error pattern from the base-call-error rates grouped according to base-call-error types. FIG. 4 and the corresponding discussion provide additional detail regarding the variation-source-identification system 106 detecting the base-call-error pattern in accordance with one or more embodiments. As shown in FIG. 4, the variation-source-identification system 106 determines the base-call-error type and neighboring nucleotide bases for each incorrect nucleotide-base call. The variation-source-identification system 106 further groups the incorrect nucleotide-base calls according to neighboring nucleotide bases and base-call-error type and detects base-call-error patterns based on the grouped incorrect nucleotide-base calls.

As illustrated in FIG. 4, the series of acts 400 includes the act 402 of determining base-call error rates grouped according to base-call-error types and different neighboring nucleotide bases. As previously mentioned, specific base-call-error types such as A→T may be greater contributors to the general error rate than other base-call-error types. Additionally, though confusion matrix data may show particular base-call-error types have higher error rates, flanking nucleotides may also be major contributors to the general error rate. Generally, the variation-source-identification system 106 determines groups of the base-call-error rates and determines the base-call-error patterns based on the determined groups. As mentioned previously, a base-call-error type can include determining a specific type of incorrect nucleotide-base call instead of a specification type of correct nucleotide-base call. For instance, the variation-source-identification system 106 determines a base-call-error type of A→T indicating an incorrect nucleotide-base call T for the actual base A. The variation-source-identification system 106 determines the base-call-error type for each incorrect nucleotide-base call and groups the base-call-error rates according to the base-call-error types.

Additionally, or alternatively, the variation-source-identification system 106 groups the base-call-error rates according to differing neighboring nucleotide bases. In particular, the variation-source-identification system 106 determines a group for each combination of possible flanking upstream and downstream nucleotide bases. In some embodiments, the variation-source-identification system 106 determines groups based on a single upstream and a single downstream neighboring nucleotide base. For example, and as illustrated in FIG. 4, the variation-source-identification system 106 can determine a group comprising incorrect nucleotide-base calls flanked by an upstream T and a downstream T (i.e., T_T). In one example, the variation-source-identification system 106 determines groups based on neighboring nucleotide bases independent of the base-call-error type. In other embodiments, the variation-source-identification system 106 determines groups based on a combination of both base-call-error types and neighboring nucleotide bases.

To illustrate, the variation-source-identification system 106 can assign base-call error rates of a particular base-call-error type to groups according to neighboring nucleotide bases. For example, the variation-source-identification system 106 groups base-call error rates of the A→T base-call-error type according to the neighboring nucleotide bases. By grouping base-call error rates according to both base-call-error types and differing neighboring nucleotide bases, the variation-source-identification system 106 generates more detailed groups of base-call error rates.

While FIG. 4 illustrates grouping base-call error rates according to two neighboring nucleotide bases—one upstream base and one downstream base—the variation-source-identification system 106 may group base-call error rates according to more neighboring nucleotide bases. For example, the variation-source-identification system 106 can delineate more groups by taking into consideration four neighboring nucleotide bases (e.g., two upstream bases and two downstream bases), six neighboring nucleotide bases (e.g., three upstream bases and three downstream bases), or more.

As further illustrated in FIG. 4, the variation-source-identification system 106 performs the act 404 of detecting the base-call-error pattern from the grouped base-call-error rates. Generally, the base-call-error pattern includes a set of normalized nucleotide specific errors that move or occur together. More specifically, the variation-source-identification system 106 tracks which groups of base-call-error rates increase in concordance with each other. For example, in one or more embodiments, the variation-source-identification system 106 simply uses the normalized error rates grouped according to base-call-error type and/or neighboring nucleotide bases as the base-call-error pattern.

The three-dimensional chart illustrated in FIG. 4 represents an example base-call-error pattern. As illustrated, the variation-source-identification system 106 identifies greater numbers of base-call-error rates or Single Nucleotide Variants (SNV) in C→A when flanked by T_A and A→C when flanked by C_T groupings.

In some embodiments, the variation-source-identification system 106 determines a threshold error value for counting base-call-error rates as part of a base-call-error pattern. Generally, sequencing runs are subject to a baseline error. In some examples, the variation-source-identification system 106 determines to disregard the baseline error in its detection of base-call-error patterns by utilizing a threshold error value. In particular, in some embodiments, the variation-source-identification system 106 utilizes an expected baseline error to determine the threshold error value. The variation-source-identification system 106 determines the expected baseline error based on user input by utilizing quality data from the sequencing system or other error prediction methods.

In one or more examples, the variation-source-identification system 106 determines the threshold error value by determining a magnification of the expected baseline error. For example, in at least one embodiment, the variation-source-identification system 106 determines that the threshold error value is 2× the expected baseline error. In some embodiments, the variation-source-identification system 106 utilizes the same threshold error value across all groups of base-call-error rates. For example, the variation-source-identification system 106 determines that the expected baseline error rate is 0.1% and accordingly sets the threshold error value as 0.2% error rate. Accordingly, the variation-source-identification system 106 disregards base-call-error rates below 0.2% when detecting the base-call-error pattern. In some embodiments, the variation-source-identification system 106 utilizes a different magnification of the expected baseline error as the threshold error value. For instance, the variation-source-identification system 106 may magnify the expected baseline error by 2.5×, 3×, etc., to determine the threshold error value. In some embodiments, the variation-source-identification system 106 pre-determines the expected baseline error rate based on historical sequencing runs that sequence a reference genome, such as PhiX.

In some embodiments, the variation-source-identification system 106 determines a plurality of threshold error rates corresponding to each group of base-call-error rates. The variation-source-identification system 106 determines expected baseline errors for each group of base-call-error rates. For example, the variation-source-identification system 106 can determine expected baseline errors for each base-call-error type. Additionally, or alternatively, the variation-source-identification system 106 can determine expected baseline errors for differing neighboring nucleotide bases. To illustrate, the variation-source-identification system 106 can determine the baseline error rate for A→T equals 0.1% while the baseline error rate for T→C equals 0.05%. Accordingly, the variation-source-identification system 106 determines the threshold error value for A→T equals 0.2% (0.1%×2) and the threshold error value for T→C equals 0.1% (0.05%×2). As mentioned, the variation-source-identification system 106 can determine additional threshold error values for groups of neighboring nucleotide bases or combinations of base-call-error type and neighboring nucleotide bases.

FIG. 4 illustrates the variation-source-identification system 106 detecting a base-call-error pattern in accordance with one or more embodiments. As mentioned, the variation-source-identification system 106 identifies a sample-base-call-error pattern that correlates to the base-call-error pattern. Sample-base-call-error patterns are from sample sequencing runs with known manufacturing data. In some embodiments, by analyzing the sample sequencing runs and manufacturing data, the variation-source-identification system 106 can predict failure sources corresponding with the sample sequencing runs.

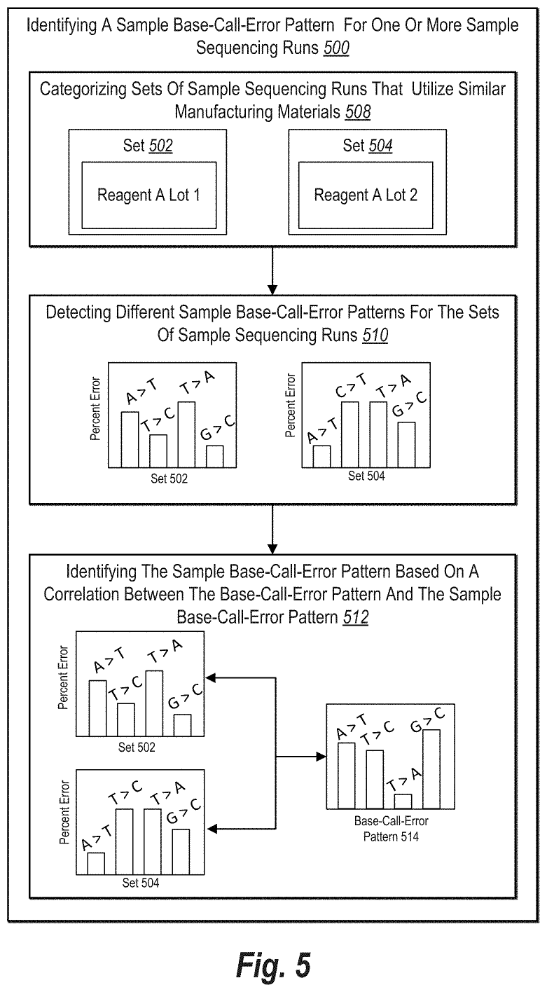

FIG. 5 and the corresponding discussion describe the variation-source-identification system 106 identifying a sample-base-call-error pattern for one or more sample sequencing runs in accordance with one or more embodiments. As illustrated in FIG. 5, the variation-source-identification system 106 performs an act 500 of identifying a sample base-call-error pattern for one or more sample sequencing runs. In particular, the variation-source-identification system 106 identifies a sample-base-call-error pattern for one or more sample sequencing runs that utilize one or more sequencing pipelines corresponding to the sequencing pipeline. More specifically, the variation-source-identification system 106 searches through sample-base-call-error patterns corresponding to a particular sequencing pipeline. For example, if the variation-source-identification system 106 determines that the base-call-error rates are generated by a first sample sequencing pipeline utilizing model x of a sequencing device and a series y of consumable product, the variation-source-identification system 106 identifies the one or more sample base-call-error patterns from sample sequencing runs utilizing the model x (or similar model) of the sequencing device and the series y (or similar model) of the consumable product. To illustrate, to identify such a sample base-call-error pattern, the variation-source-identification system 106 performs a series of acts including an act 508 of categorizing sets of sample sequencing runs that utilize similar manufacturing materials, an act 510 of detecting different sample base-call-error patterns for the sets of sample sequencing runs, and an act 512 of identifying the sample base-call-error pattern based on a correlation between the base-call-error pattern and the sample base-call-error pattern.

FIG. 5 illustrates the variation-source-identification system 106 performing the act 508 of categorizing sets of sample sequencing runs that utilize similar manufacturing materials. Generally, as part of identifying failure sources within sample sequencing runs, the variation-source-identification system 106 defines sets of sample sequencing runs with similar manufacturing materials. As mentioned, the variation-source-identification system 106 can identify various types of failure sources within a sequencing pipeline, including hardware, chemistry, and software. Hardware entails both the equipment that makes up sequencing devices as well as some consumables, such as a nucleotide-sample slide (e.g., flow cell), that the sequencing devices utilize during sequencing. Chemistry includes reagents and interactions between reagents or between consumables and reagents—as well as between reagents and hardware part of a sequencing device. Software comprises programs and operating information utilized by the sequencing pipeline. For instance, the software can include a sequence-analysis software, such as DRAGEN offered by Illumina, Inc.

In some embodiments, the variation-source-identification system 106 identifies sets of sample sequencing runs that utilize similar consumables. For example, and as illustrated in FIG. 5, the variation-source-identification system 106 defines a set 502 of sample sequencing runs and a set 504 of sample sequencing runs. As illustrated, the set 502 includes sample sequencing runs that utilize reagent A from lot 1 whereas the set 504 includes sample sequencing runs that utilize reagent A from lot 2. While FIG. 5 illustrates the variation-source-identification system 106 categorizing sets based on reagents, the variation-source-identification system 106 can categorize sets based on sample sequencing runs that utilize similar equipment or software.

As part of categorizing sets, the variation-source-identification system can assign a single sample sequencing run to several sets. For example, the variation-source-identification system 106 can assign a particular sample sequencing run to the set 502 based on determining that the particular sample sequencing run utilizes reagent A from lot 1. The variation-source-identification system 106 can further assign the particular sample sequencing run to a second set based on the particular sample sequencing run utilizing a nucleotide-sample-slide from a particular lot.

As further illustrated in FIG. 5, the variation-source-identification system 106 performs the act 510 of detecting different sample base-call-error patterns for the sets of sample sequencing runs. Generally, the variation-source-identification system 106 performs acts similar to those portrayed in FIGS. 3-4 to detect different sample base-call-error patterns for the sets of sample sequencing runs. In some embodiments, the variation-source-identification system 106 generates a sample base-call-error pattern for each sample sequencing run within a set of sample sequencing runs and aggregates the sample base-call-error patterns. In some embodiments, the variation-source-identification system 106 can determine statistically significant sample error rates across the sample sequencing runs within a set of sample sequencing runs.

For example, and as illustrated in FIG. 5, the variation-source-identification system 106 determines sample base-call-error patterns for the set 502 and the set 504. FIG. 5 illustrates the variation-source-identification system 106 generating sample base-call-error patterns that group sample base-call-error rates based on base-call-error types. In some embodiments, the variation-source-identification system 106 groups sample base-call-error rates based on base-call-error type and/or neighboring nucleotide bases. FIG. 6A and the corresponding discussion provide additional detail relating to detecting different sample base-call-error patterns for the sets of sample sequencing runs.

As further illustrated in FIG. 5, the variation-source-identification system 106 performs the act 512 of identifying the sample base-call-error pattern based on a correlation between the base-call-error pattern and the sample base-call-error pattern. In particular, the act 512 comprises identifying the sample base-call-error pattern from among the different sample base-call-error patterns for the sets of sample sequencing runs based on the correlation between the base-call-error pattern and the sample base-call-error pattern. In some embodiments, the variation-source-identification system 106 identifies sample base-call-error patterns that are the same as the base-call-error pattern. In some embodiments, the variation-source-identification system 106 identifies one or more sample base-call-error patterns that are similar to the base-call-error pattern.

To illustrate, in FIG. 5, the variation-source-identification system 106 identifies similarities between the set 502 and the set 504 with a base-call-error pattern 514. For example, the variation-source-identification system 106 detecting the set 502 for including an elevated A→T percent error and detecting the set 504 for including elevated an elevated T→C percent error that correspond with the elevated A→T and T→C percent errors of the base-call-error pattern 514.

Though FIG. 5 illustrates the variation-source-identification system 106 comparing base-call-error-patterns for the sets of sample sequencing runs, in some embodiments, the variation-source-identification system 106 compares the base-call-error pattern 514 with failure-specific sample-base-call-error patterns or individual sample base-call-error patterns. In particular, to determine failure-specific sample-base-call-error patterns, the variation-source-identification system 106 generates a sample-base-call-error pattern corresponding to a single failure mode. In particular, in some embodiments, the variation-source-identification system 106 identifies failure-specific sample-base-call-error rates that increase with particular failure sources. For example, the variation-source-identification system 106 can determine that an increase in sample-base-call-error rates of the A→C base-call-error type with T_T as neighboring nucleotide bases directly correlate with flow cell lot issues. In some embodiments, the variation-source-identification system 106 generates the failure-specific sample-base-call-error patterns by utilizing a statistical model described in additional detail below in the paragraphs corresponding to FIG. 6A.

Accordingly, in addition, or in the alternative, to identifying sample base-call-error patterns from sets of sample sequencing runs that correspond to the base-call-error pattern 514, the variation-source-identification system 106 identifies one or more failure-specific sample-base-call-error patterns that correspond to the base-call-error pattern 514. For example, based on determining that the base-call-error pattern 514 includes an elevated percent error of an A→T base-call-error rate, the variation-source-identification system 106 identifies the corresponding A→T failure-specific sample base-call-error pattern. Similarly, the variation-source-identification system 106 can identify a second failure-specific sample-base-call-error pattern comprising a combination of elevated T→C and G→C percent errors corresponding with the elevated T→C and G→C base-call-error rate within the base-call-error pattern 514.

In some embodiments, the variation-source-identification system 106 identifies an individual sample-base-call-error pattern that corresponds to the base-call-error pattern 514. In particular, instead of aggregating sample base-call-error-patterns for sample sequencing runs within a set, the variation-source-identification system 106 selects an individual base-call-error pattern that corresponds to the base-call-error pattern 514.

In one or more embodiments, the variation-source-identification system 106 performs the act 512 of identifying the sample base-call-error pattern based on a correlation between the base-call-error pattern and the sample base-call-error pattern by utilizing a machine learning model to identify sample base-call-error patterns that are similar to the base-call-error pattern 514. For example, the variation-source-identification system 106 can utilize a clustering algorithm such as K-means clustering, multivariate k-means clustering, or other types of clustering algorithms. In one example, the variation-source-identification system 106 utilizes sample-base-call error patterns to train a clustering algorithm. In particular, the variation-source-identification system 106 may utilize the sample base-call-error patterns to predict which sample sequencing runs resulted in similar sample failure sources. The variation-source-identification system 106 applies the trained clustering algorithm to base-call-error patterns to identify which one or more sample base-call-error patterns are most similar to the base-call-error pattern.

In some embodiments, the variation-source-identification system 106 utilizes user input to further train the machine learning model described above. For example, the variation-source-identification system 106 can provide, for display to a user, an option to confirm a predicted failure source. Based on a data indication from a client device confirming a predicted failure source as the failure source, the variation-source-identification system 106 can further validate the probability associated with the failure source. By contrast, based on receiving a denial of a predicted failure source, the variation-source-identification system 106 can adjust parameters of the machine learning model to provide more accurate predictions (e.g., contribution metrics) in the future.

In some embodiments, and as illustrated in FIG. 5, the variation-source-identification system 106 identifies an existing sample base-call-error pattern for the one or more sample sequencing runs. In particular, the variation-source-identification system 106 can identify an existing sample base-call-error pattern that is the same as, or similar to, the base-call-error pattern from a repository of sample base-call-error patterns. More specifically, the variation-source-identification system 106 can utilize a clustering algorithm described above to determine a similar existing sample base-call-error pattern from the repository of base-call-error patterns. For example, the variation-source-identification system 106 can determine that the base-call-error pattern indicates elevated error rates of a C→G base-call error type with C_G neighboring nucleotides and an A→T base-call-error type with A T neighboring nucleotides. The variation-source-identification system 106 can identify a first existing sample base-call-error pattern having the same elevated error rates of the C→G base-call error type with C_G neighboring nucleotides and a second existing sample base-call-error pattern having similar elevated error rates of the A→T base-call error type with A_T neighboring nucleotides. Accordingly, the A→T base-call error type with A_T neighboring nucleotides determines a correlation between the base-call-error pattern and the first and second existing sample base-call-error patterns.

As part of performing the act 512 of identifying the sample base-call-error pattern based on a correlation between the base-call-error pattern and the sample base-call-error pattern, in some cases, the variation-source-identification system 106 filters out sample base-call-error patterns that do not correlate with the base-call-error pattern. For example, in some embodiments, based on determining that the base-call-error pattern corresponds to one or more sample base-call-error patterns, the variation-source-identification system 106 filters out a set of dissimilar sample base-call-error patterns that do not correspond to the one or more sample base-call-error pattens. By excluding the dissimilar sample base-call-error patterns, the variation-source-identification system 106 can analyze remaining sample base-call-error patterns for a best correspondence or match to the base-call-error pattern in question.

Additionally, or alternatively, the variation-source-identification system 106 detects a new sample base-call-error pattern for the one or more sample sequencing runs. In particular, in some embodiments, the variation-source-identification system 106 determines that the base-call-error pattern does not correspond to an existing sample base-call-error pattern. In such cases, the variation-source-identification system 106 can identify a new sample base-call-error pattern based on the base-call-error pattern. For example, the variation-source-identification system 106 can designate the base-call-error pattern as a new sample base-call-error pattern and utilize a statistical model to analyze the new sample base-call-error pattern with manufacturing data corresponding to the new sample base-call-error pattern. In other embodiments, the variation-source-identification system 106 detects the new sample-base-call-error pattern by aggregating a combination of sample-base-call-error patterns that are similar to the base-call-error pattern.

Generally, and as described previously, the variation-source-identification system 106 determines a correlation between one or more sample base-call-error patterns and a base-call-error pattern. The variation-source-identification system 106 further identifies failure sources for the base-call-error pattern by identifying failure sources corresponding to the one or more sample-base-call-error patterns. While FIG. 5 and the corresponding paragraphs describe the variation-source-identification system 106 identifying one or more sample base-call-error patterns that correspond to a base-call-error pattern, FIGS. 6A-6C and the corresponding discussion describe the variation-source-identification system 106 determining a correlation between sample base-call-error patterns and failure sources. As mentioned, the variation-source-identification system 106 determines contribution metrics indicating probabilities of sequencing-pipeline materials contributing to base-call errors from a sequencing pipeline.

FIGS. 6A-6C and the corresponding paragraphs provide detail regarding the variation-source-identification system 106 determining failure sources corresponding to sample base-call-error patterns and/or base-call-error patterns in accordance with one or more embodiments. Generally, FIGS. 6A-6C illustrate inputs that the variation-source-identification system 106 processes utilizing a statistical model 614 to determine contribution metrics 622 indicating probabilities of sequencing-pipeline materials 620 contributing to base-call errors from a sequencing pipeline. As an overview, the variation-source-identification system 106 utilizes the statistical model 614 to process sample sequencing data 616 and manufacturing data 618.

As shown in FIG. 6A, the variation-source-identification system 106 processes sample sequencing data 616 to use as input into the statistical model 614. In particular, FIG. 6A illustrates several acts for processing the sample sequencing data 616 including an act 602 of aggregating sample nucleotide-fragment reads, an act 604 of determining normalized sample error rates, and an act 608 of grouping the normalized sample error rates according to base-call-error types and different neighboring nucleotide bases. FIG. 6A further illustrates several acts for processing the manufacturing data 618. In particular, the variation-source-identification system 106 performs an act 610 of truncating manufacturing identification data and an act 612 of generating a set of sequencing runs by grouping a threshold number of sequencing runs.

As indicated above, the variation-source-identification system 106 can utilize sequencing devices to generate sample nucleotide-base-calls for a reference genome. In some embodiments, prior to performing the act 602 of aggregating sample nucleotide-fragment reads, the variation-source-identification system 106 performs additional pre-processing acts to improve the quality of the sample sequencing data 616. For example, the variation-source-identification system 106 can perform an additional act of identifying passing sample sequencing runs and an additional act of removing alignment errors. In some embodiments, sample sequencing runs are part of quality assurance measures to ensure that sequencing devices perform a threshold error standard. Accordingly, some sample sequencing runs from particular sequencing devices contain error rates that exceed a threshold error standard. Thus, in some embodiments, the variation-source-identification system 106 removes non-passing sample sequencing runs to provide a more realistic representation of normal sequencing variation.

As part of performing the act 602 of aggregating the sample nucleotide-fragment reads, in some embodiments, the variation-source-identification system 106 processes data from a variant call file, such as Variant Call Format (VCF) file. Generally, a variant call file contains information about variants found at specific positions or genomic coordinates in a reference genome. Thus, as part of performing the act 602, the variation-source-identification system 106 aggregates VCF data for a read one forward (R1F), a read one reverse (R1R), a read two forward (R2F), and a read two reverse (R2R) for each sequencing run. The aggregated VCF data can provide a representation of normal sequencing variation. By aggregating the VCF data for the various reads, in some cases, the variation-source-identification system 106 generates VCF data for an aggregated read one (R1) and an aggregated read two (R2).

Additionally, and as previously mentioned, the variation-source-identification system 106 sometimes performs an additional pre-processing step of removing alignment errors within the sample sequencing data 616. In particular, the variation-source-identification system 106 can identify alignment errors that occur above a threshold variant frequency and remove the identified alignment errors. For example, based on determining that an alignment error occurs above a 60% threshold variant frequency, the variation-source-identification system 106 removes the reference genome alignment errors.

As further illustrated in FIG. 6A, the variation-source-identification system 106 performs the act 602 of aggregating sample nucleotide-fragment reads. Generally, the variation-source-identification system 106 aggregates multiple reads from a single sequencing run to consolidate sample sequencing data. In particular, sequencing systems typically determine thousands to millions of nucleotide-fragment reads from oligonucleotides extracted from the reference genome. Furthermore, the sequencing systems may also determine both forward and reverse nucleotide-fragment reads. For example, in some embodiments, sequencing systems generates a R1F, a R1R, a R2F, and R2R for each sample sequencing run.

After determining nucleotide-fragment reads, the variation-source-identification system 106 aligns the nucleotide-fragment reads with the reference genome. More specifically, the variation-source-identification system 106 aligns the R1F and the R2F reads to the forward portion of the reference genome, and the variation-source-identification system 106 aligns the R1R and the R2R reads to the reverse complement of the reference genome. In some embodiments, the variation-source-identification system 106 combines the forward and reverse reads to further simplify data.

As suggested by FIG. 6A, after aligning the nucleotide-fragment reads, the variation-source-identification system 106 analyzes the aligned nucleotide-fragment reads to determine sample nucleotide-base calls. The variation-source-identification system 106 can further compare the sample nucleotide-base calls with reference bases of a reference genome to identify correct and incorrect sample nucleotide-base calls. For example, in some embodiments, the variation-source-identification system 106 utilizes the confusion matrix illustrated in FIG. 3 to determine sample nucleotide-specific error rates.

As further illustrated in FIG. 6A, the variation-source-identification system 106 performs the act 604 of determining normalized sample error rates. Generally, the variation-source-identification system 106 may utilize a confusion matrix to generate sample base-call-error rates. The variation-source-identification system 106 normalizes the sample base-call-error rates in a similar manner in how the variation-source-identification system 106 normalizes base-call-error rates as described above in relation to FIG. 3. In some implementations, the variation-source-identification system 106 determines that a percent error equals the count of a specific error divided by the count of a correct call. Consistent with the disclosure above explaining how the variation-source-identification system 106 normalizes base-call-error rates, the variation-source-identification system 106 may determine normalized sample base-call-error rates for particular base-call-error types and/or neighboring nucleotide bases.

As further shown in FIG. 6A, after performing the act 604 of determining the normalized sample error rates, the variation-source-identification system 106 performs the act 608 of grouping the normalized sample error rates according to base-call-error types and different neighboring nucleotide bases. In particular, the variation-source-identification system 106 generates sample base-call-error patterns by grouping the normalized sample error rates in a similar manner to how the variation-source-identification system 106 groups normalized base-call-error rates as described above in relation to FIG. 4. In one or more embodiments, the variation-source-identification system 106 utilizes the sample base-call-error patterns as input into the statistical model 614.

FIG. 6A illustrates an example series of acts by which the variation-source-identification system 106 pre-processes and processes the sample sequencing data 616 for analysis by the statistical model 614. In particular, FIG. 6A illustrates utilizing normalized sample error rates and groups of the sample error rates as input into the statistical model 614. Additionally, or alternatively, the variation-source-identification system 106 utilizes other sample sequencing data as input into the statistical model 614. To illustrate, in some embodiments, the variation-source-identification system 106 can access sequencing run error rates, quality scores, alignment metrics, read depth, and other primary or secondary metrics obtained from the sequencing pipeline.

As further illustrated in FIG. 6A, the variation-source-identification system 106 utilizes the statistical model 614 to analyze the manufacturing data 618. Generally, the variation-source-identification system 106 processes the manufacturing data 618 to identify sets of sample sequencing runs that utilize similar manufacturing materials, other hardware, chemistry, and/or software. Manufacturing data generally includes data indicating the identity and various properties of materials, hardware, chemistry, and/or software used in sequencing runs. In particular, manufacturing data can include the general purpose, identity, manufacture number, or other identifying information associated with a piece of hardware, consumable, or software. For example, manufacturing data can comprise a lot number or a date of production or release associated with a reagent, part, or software version. In some embodiments, the variation-source-identification system 106 processes the manufacturing data 618 by performing the act 610 of truncating manufacturing identification data and the act 612 of generating a set of sequencing runs by grouping a threshold number of sequencing runs.

In some embodiments, and as illustrated in FIG. 6A, the variation-source-identification system 106 performs the act 610 of truncating manufacturing identification data. In many instances, failure sources are localized to manufacturing materials from the same or similar lots or manufacturing materials produced within the same or similar timeframe. For example, a production error that is evident in one manufacturing material has likely impacted similar manufacturing materials from the same production lot. One method by which the variation-source-identification system 106 identifies similar manufacturing materials is by performing the act 610 of truncating manufacturing identification data. Manufacturing identification data can include barcode IDs or other manufacturing identification codes. As illustrated, the variation-source-identification system 106 can truncate a seven-digit manufacturing identification number to a four-digit truncated manufacturing ID.

As further illustrated in FIG. 6A, the variation-source-identification system 106 performs the act 612 of generating a set of sequencing runs by grouping a threshold number of sequencing runs. In particular, the variation-source-identification system 106 performs the act 612 by generating a set of sequencing runs by grouping a threshold number of sequencing runs that share the same truncated manufacturing identification data. As illustrated, the variation-source-identification system 106 groups sequencing runs corresponding to the manufacturing identification numbers 1234567, 1234566, 1234565, and 1234564 based on them sharing the same truncated manufacturing identification data of 1234. In some embodiments, the variation-source-identification system 106 also sets a target percentage of sequencing runs to be assigned to sets of sequencing runs. For example, the variation-source-identification system 106 may target grouping at least 80% of sequencing runs into sets containing at least ten or more sequencing runs.

FIG. 6A illustrates the variation-source-identification system 106 performing a particular series of acts for processing the manufacturing data 618 in accordance with one or more embodiments. The variation-source-identification system 106 may utilize additional or alternative methods for processing the manufacturing data 618 for entry into the statistical model 614. For instance, instead of utilizing manufacturing identification data, the variation-source-identification system 106 may generate sets of sample sequencing runs by vendor, hardware type or identification, software type or identification, or chemistry type or identification.

As illustrated in FIG. 6A, the variation-source-identification system 106 utilizes the statistical model 614 to analyze the sample sequencing data 616 and the manufacturing data 618. In particular, the variation-source-identification system 106 determines, utilizing the statistical model 614, contribution metrics indicating probabilities of sequencing-pipeline materials contributing to base-call-errors from the sequencing pipeline. In at least one embodiment, the statistical model 614 comprises a variance components model. The variation-source-identification system 106 utilizes the variance components model to generate percentages of assignable cause variations for sequencing-pipeline materials contributing the to the base-call errors. In particular, the variation-source-identification system 106 can utilize the variance components model to determine percentages that indicate probabilities that given sequencing-pipeline materials are the source of variation or other failure source.

Additionally, or alternatively, the statistical model 614 comprises other types of statistical models or algorithms. For example, in one or more embodiments, the statistical model 614 comprises boundary value analysis and equivalence partitioning testing for continuous data. More specifically, instead of truncating manufacturing identification data, the variation-source-identification system 106 can utilize whole manufacturing identification data. The variation-source-identification system 106 utilizes equivalence partitioning testing to identify equivalence partitions or groups of equivalent sequencing runs having similar sample sequencing data based on un-truncated manufacturing identification data. In some embodiments, the variation-source-identification system 106 further utilizes boundary analysis testing to test boundaries between equivalence partitions.

As further illustrated in FIG. 6A, the variation-source-identification system 106 utilizes the statistical model 614 to analyze the sample sequencing data 616 and the manufacturing data 618 associated with the sample sequencing data 616. In one or more embodiments, the variation-source-identification system 106 utilizes the statistical model 614 to analyze any other sequencing data. For example, in some embodiments, the sample sequencing data 616 represents internal quality testing data for which the manufacturing data 618 is controlled or known. The variation-source-identification system 106 may also collect sequencing data that is not sample sequencing data. For example, in some embodiments, the variation-source-identification system 106 collects sequencing data together with manufacturing data for each sequencing run utilizing a sequencing device.

FIG. 6B illustrates an example output generated by the variation-source-identification system 106 utilizing the statistical model 614. In particular, FIG. 6B illustrates example contribution metrics 622 that indicate probabilities of the sequencing-pipeline materials 620 contributing to base-call errors from the sequencing pipeline. More specifically, FIG. 6B illustrates the percentages of assignable cause variations generated by the variation-source-identification system 106 for the sequencing pipe-line materials contributing to base-call errors. In some embodiments, the variation-source-identification system 106 generates percent assignable cause variations by utilizing a variance components model. Generally, the percent assignable cause variations represent a probability that a given sequencing pipeline material is responsible for a particular base-call-error type. For example, for the error type G→A having neighboring nucleotides C_T, the variation-source-identification system 106 determines that clustering reagent HCXE2 has an impact as well as LDR (Ligase Detection Reaction), a denaturation agent. Each bar in the graph illustrated in FIG. 6B reveals probabilities that specific drivers to a particular nucleotide change along with its neighboring nucleotides.

The sequencing-pipeline materials 620 illustrated in FIG. 6B indicate various components that contribute to the sequencing pipeline. For example, the sequencing-pipeline materials 620 can include consumable products, parts of sequencing machines, or parts of nucleotide-sample slides. In some embodiments, the sequencing-pipeline materials 620 comprise additional components. Generally, the sequencing-pipeline materials 620 can comprise any part of hardware, chemistry, or software that contribute to the sequencing pipeline.

As mentioned, the variation-source-identification system 106 can generate percent assignable cause variations for sequencing pipeline materials. In some embodiments, the variation-source-identification system 106 generates a ranked list based on the percent assignable cause variations. For instance, the variation-source-identification system 106 ranks the sequencing pipeline materials from greatest percentage of assignable cause to lowest percentage. The ranking thus indicates which sequencing pipeline material has the most likely prominent correlation for shifts in errors. Furthermore, the variation-source-identification system 106 may determine one or more failure sources based on the generated percent assignable cause variations. For example, in some cases, the variation-source-identification system 106 determines a primary failure source is the sequencing pipeline material associated with the greatest percent assignable cause variation.

As described in relation to FIGS. 6A-6B, the variation-source-identification system 106 leverages the sample error rates grouped according to base-call-error type and different neighboring nucleotide bases to determine correlations between failure sources and base-call-error patterns. FIG. 6C illustrates a bar graph 624 representing the percentile occurrence of base-call errors organized by base-call-error type. Generally, the bar graph 624 demonstrates that base-call-error rates are unevenly distributed across base-call-error types. For instance, and as illustrated in FIG. 6C, base-call errors of the T→A base-call-error type occur far more frequently than base-call errors of the T→G base-call-error type. Additionally, and as illustrated in FIG. 6C, errors involving Ts are more prevalent (as seen by T→A, T→C, and A→T peaks).

As further illustrated by the shaded boxes within the bar graph 624 of FIG. 6C, base-call-error rates can also be unevenly distributed across nucleotide-fragment reads. For example, read two (R2) tends to experience more error than read one (R1), likely due to signal decay between R1 and R2. Accordingly, in some embodiments, the variation-source-identification system 106 can group normalized sample error rates according to read number (e.g., R1 and R2) in addition, or in the alternative to, grouping the normalized sample error rates according to base-call-error types and different neighboring nucleotide bases.

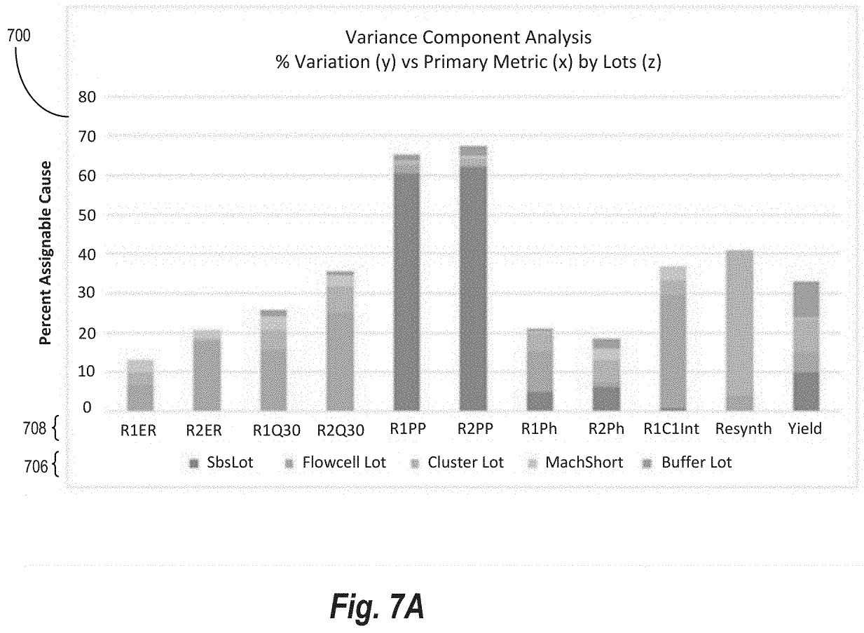

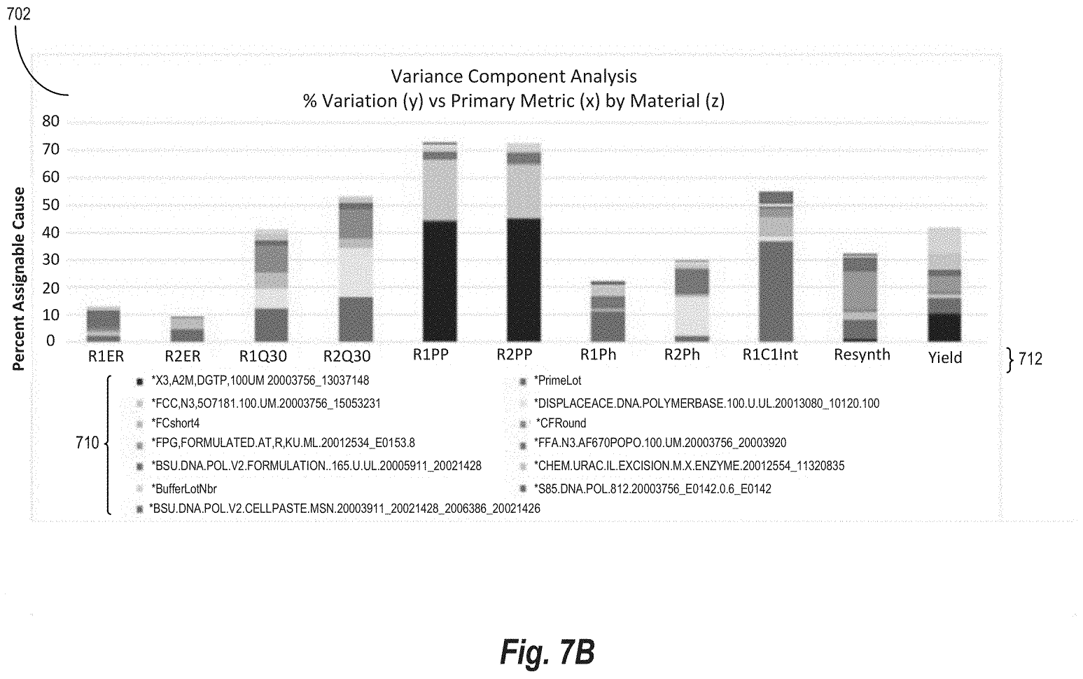

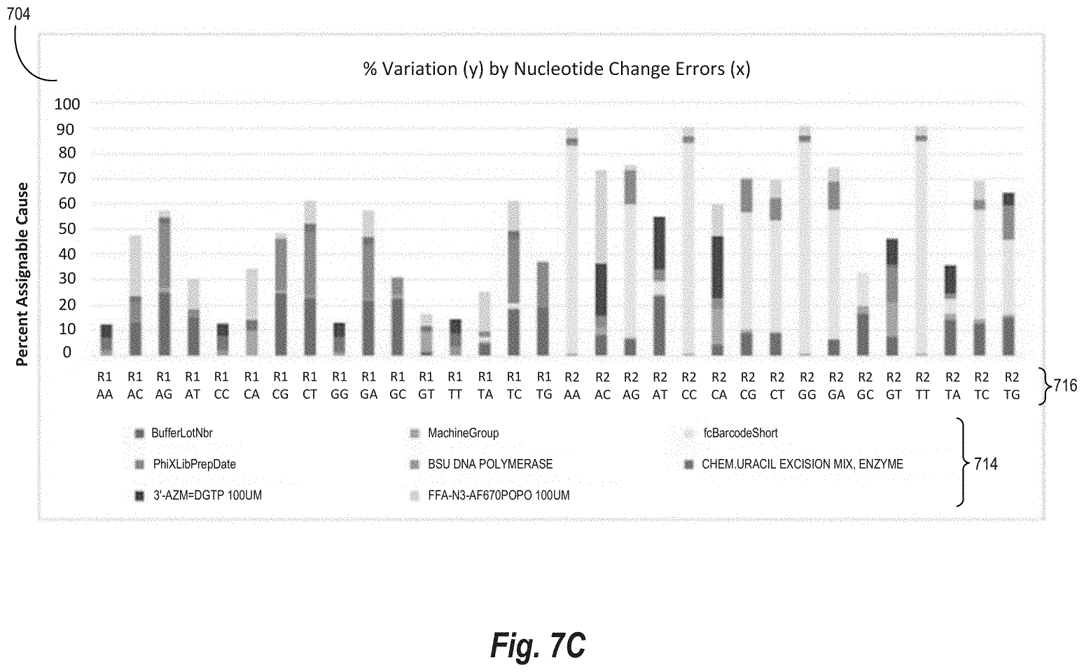

FIGS. 6A-6C illustrate the variation-source-identification system 106 utilizing a statistical model to determine contribution metrics indicating contributions of sequencing-pipeline materials to base-call errors from the sequencing pipeline in accordance with one or more embodiments. FIGS. 7A-7C illustrate a series of bar graphs that represent how the variation-source-identification system 106 utilizes one or more statistical models to narrow down failure sources in a hierarchical manner to generate contribution metrics in accordance with one or more embodiments. As a brief overview, FIG. 7A illustrates a general assembly bar graph 700 demonstrating percent assignable causes based on a general assembly analysis in accordance with one or more embodiments. FIG. 7B illustrates a sub-assembly component bar graph 702 resulting from the variation-source-identification system 106 utilizing a statistical model on a sub-assembly to provide additional detail regarding a smaller subset of potential failure sources in accordance with one or more embodiments. FIG. 7C illustrates the variation-source-identification system 106 using nucleotide specific errors (instead of simple primary metrics utilized in FIGS. 7A-7B) to generate a base-call-error type bar graph 704 in accordance with one or more embodiments.

By way of introduction to FIGS. 7A-7C, in some embodiments, the variation-source-identification system 106 can identify several hundreds of variables or potential failure sources within manufacturing data. The variation-source-identification system 106 can process the hundreds of variables in a hierarchical manner that is more efficiently analyzed by a statistical model, such as VCA. In some embodiments, statistical models can accurately and efficiently process a set of potential failure sources at a time. For example, a statistical model may be limited to processing thirty-two potential failure sources at a time. Accordingly, the variation-source-identification system 106 may begin the analysis of high-level general assembly failure sources (capped at thirty-two potential failure sources) and then analyze detailed sub-assembly raw materials (again capped at thirty-two potential failure sources). FIGS. 7A-7C illustrate this hierarchical approach in accordance with one or more embodiments. While FIGS. 7A-7C include percent assignable causes generated by the variation-source-identification system 106 utilizing VCA, the variation-source-identification system 106 may utilize alternative statistical models to analyze potential failure sources in a hierarchical manner.

In particular, FIG. 7A illustrates the general assembly bar graph 700 representing percent assignable causes attributable to potential general assembly failure sources 706 for variations in primary metrics 708. As illustrated in FIG. 7A, the variation-source-identification system 106 utilizes VCA to process the potential general assembly failure sources 706. For example, the potential general assembly failure sources 706 includes SBS lot, nucleotide-sample slide (e.g., FlowCell) lot, cluster lot, Mach Short, and buffer lot. In other embodiments, the variation-source-identification system 106 utilizes VCA to process other potential general assembly failure sources, such as general software or computing failure sources and sequencing device parts.

As further illustrated in FIG. 7A, the variation-source-identification system 106 determines percent assignable causes of variation in primary metrics 708 associated with the potential general assembly failure sources 706. For example, and as illustrated in FIG. 7A, the variation-source-identification system 106 determines the potential general assembly failure sources 706 that are most probable causes for variations in the primary metrics 708. In some cases, the primary metrics 708 comprise, for R1 and R2, error rate (ER), Phred quality score (Q30), pre-phasing (PP), phasing (Ph), channel intensity (CnInt), resynthesis (Resynth), and yield. In other embodiments, the variation-source-identification system 106 generates percent assignable cause for different primary metrics, including, but not limited to, the number of clusters, number of cycles that have been error rated, the percentage of clusters passing filtering, the density of clusters, the number of tiles, and other primary metrics. In yet other embodiments, and as described below in relation to FIG. 7C, the variation-source-identification system 106 generates percent assignable causes for secondary metrics, including base-call-error type and neighboring nucleotide bases.

The variation-source-identification system 106 evaluates the potential general assembly failure sources 706 to determine which are causing the largest source of variation for the sequencing variable of interest from among the primary metrics 708. As illustrated in FIG. 7A, the variation-source-identification system 106 determines that SBS lot impacts pre-phasing the most while cluster lot impacts resynthesis the most. As further depicted in FIG. 7A, flow cell lot disproportionately impacts intensity, error rate, Phred score, and phasing. The variation-source-identification system 106 can further analyze any one of the potential general assembly failure sources 706 to further evaluate potential sub-assembly failure sources. For example, the variation-source-identification system 106 may break down the flow cell potential general assembly failure source into sub-assembly failure sources.

In particular, and as mentioned previously, the variation-source-identification system 106 can further analyze any potential general assembly failure source to evaluate its sub-assembly failure sources. In some cases, the variation-source-identification system 106 disaggregates the flow cell potential general assembly failure source into the following sub-assembly failure sources: a reagent cartridge lot, glass lot, plastic lot, primer lot, hydrogel lot, etc. To do so, the variation-source-identification system 106 holds (or sets as controls) other assembly variables at a high level to more specifically identify variability stemming from potential sub-assembly failure sources. For example, the variation-source-identification system 106 analyzes sequencing runs in which the SBS lot, cluster lot, machshort, and buffer lot are found to have little to no contribution to base call errors—then analyzes the potential sub-assembly failure sources. In some embodiments, the variation-source-identification system 106 generates a sub-assembly bar graph similar to the general assembly bar graph 700 but indicating potential sub-assembly failure sources.

By utilizing a statistical model, the variation-source-identification system 106 can analyze at a more granular level by analyzing potential sub-assembly failure sources to identify specific contributions of sub-assembly components. For example, the variation-source-identification system 106 can utilize VCA to evaluate reagent cartridge sub-assembly-specific contributions. The variation-source-identification system 106 holds (or sets as controls) other sub-assembly variables at a high level to more precisely identify variability stemming from sub-assembly components. For example, FIG. 7B illustrates the variation-source-identification system 106 evaluating potential sub-assembly component failure sources 710 for the primary metrics 712. More specifically, FIG. 7B illustrates a sub-assembly component bar graph 702 reflecting percent assignable cause variations for reagent cartridge component contributions.

As noted above, FIGS. 7A-7B illustrate the variation-source-identification system 106 utilizing VCA to generate percent assignable cause variations for potential failure sources on primary metrics such as error rate, Q30 scores, etc. In some embodiments, the variation-source-identification system 106 utilizes VCA to measure contributions of potential failure sources for other metrics, including nucleotide-specific errors. FIG. 7C illustrates the variation-source-identification system 106 determining contributions of various potential failure sources on variations in nucleotide-specific errors. In particular, FIG. 7C illustrates a base-call-error type bar graph 704 indicating contributions of potential failure sources 714 to variations in secondary metrics 716.

As illustrated in FIG. 7C, the variation-source-identification system 106 tests the potential failure sources 714 across all general assembly failure sources with the greatest or highest contributions to base-call-error rates. As illustrated in the base-call-error type bar graph 704, the potential failure sources 714 include buffer lot number (BufferLotNbr); PhiX library preparation date (PhiXLibPrepDate); machine group; flow cell bar code (fcBarcodeShort); and consumables including reagents, enzymes, nucleotide structures, etc. The secondary metrics 716 measured in FIG. 7C include the read number (R1 or R2) as well as the base-call-error type. For example, AC indicates a base-call-error type A→C, AG indicates the base-call-error type A→G, etc.

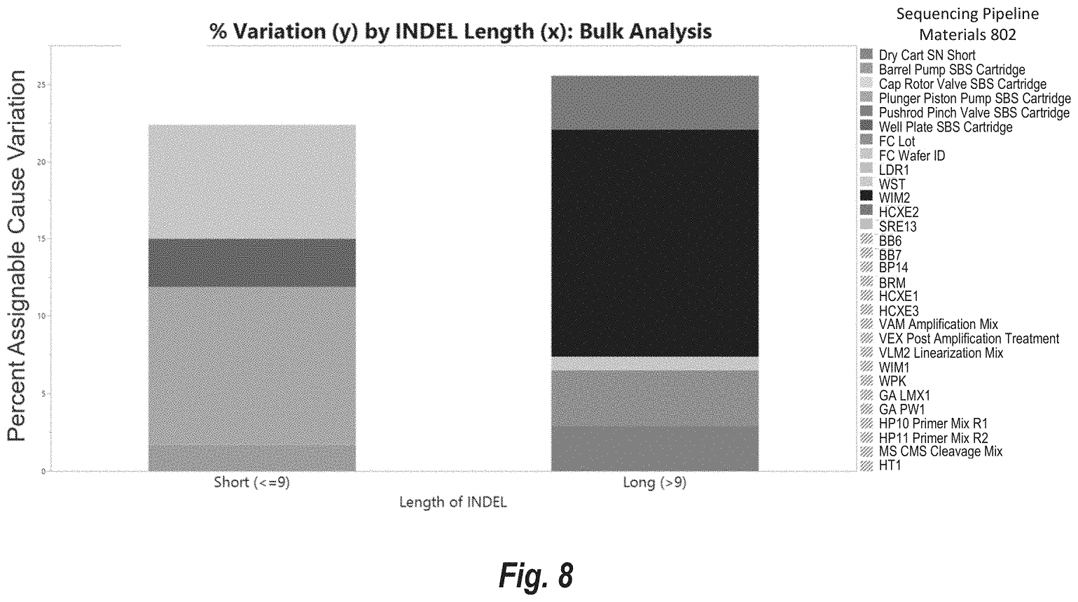

As mentioned previously, the variation-source-identification system 106 may utilize different types of sample sequencing data together with manufacturing data to determine contribution metrics. FIG. 8 illustrates an example embodiment in which the variation-source-identification system 106 utilizes insertion or deletion (INDEL) lengths as the sequencing data to determine contribution metrics indicating contributions of sequencing-pipeline materials to base-call errors from the sequencing pipeline.

Generally, in addition to driving variation in base-call-error rates, sequencing pipeline materials may also drive variation in INDEL lengths. Accordingly, the variation-source-identification system 106 may utilize a statistical model to analyze INDEL lengths and determine percent assignable cause variations for sequencing pipeline materials 802 based on INDEL lengths detected in sequencing pipelines. For instance, as illustrated in FIG. 8, shorter INDELs, where segments being inserted or deleted are less than or equal to nine nucleotides, are primarily driven by hardware and fluidics. More specifically, flow cell and fluidic differences including barrel pump, plunger, and well plate sequencing pipeline materials have greater probabilities of contributing to variations in INDEL lengths. In contrast, longer INDELs, where the inserted or deleted segment is greater than nine nucleotides, is more heavily driven by flow cell and incorporation mixes. More specifically, an SBS dye reagent (e.g., WIM 2) and a clustering reagent (e.g., HCXE2) are more prominent drivers in contributing to longer INDEL variations.

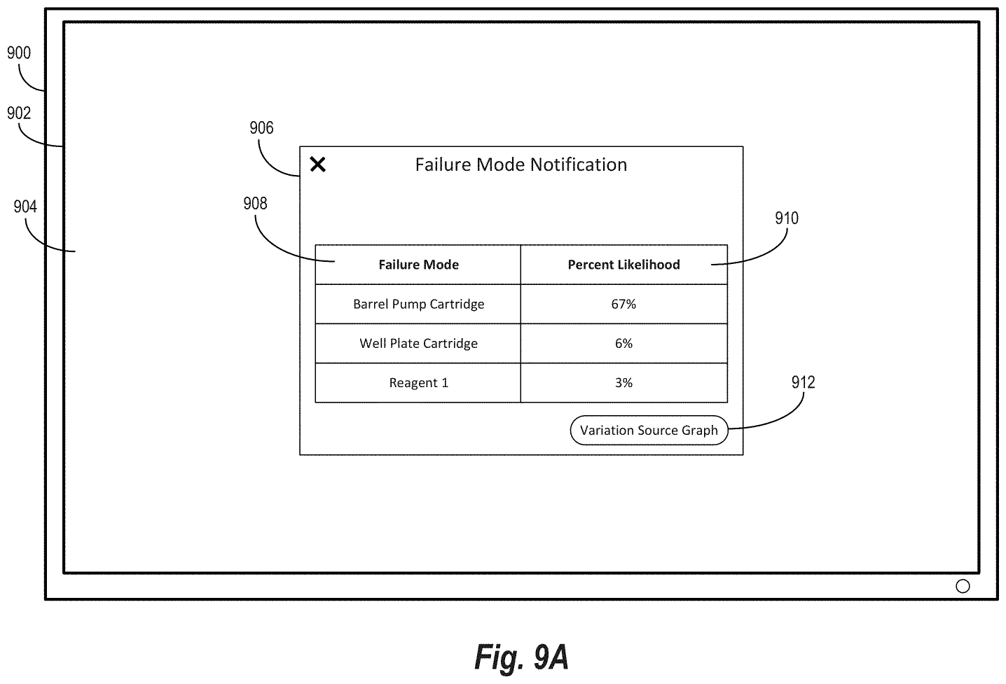

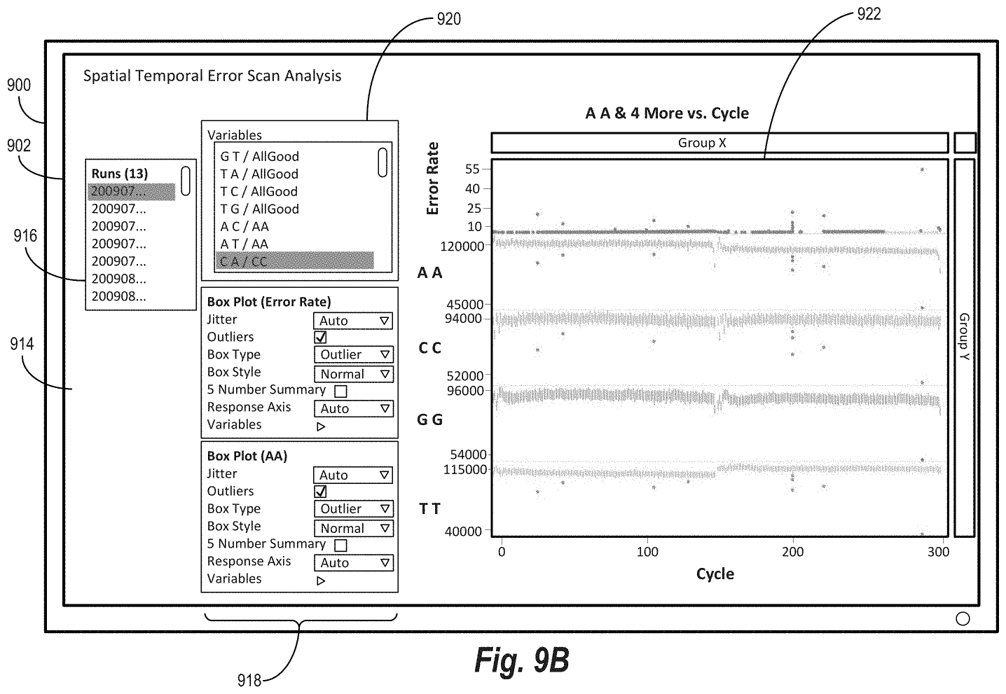

As indicated above, in some embodiments, the variation-source-identification system 106 provides, for display on a computing device associated with a sequencing pipeline, a notification indicating one or more failure sources. FIGS. 9A-9B illustrate a series of graphical user interfaces including a failure mode notification and additional information regarding identified failure sources. As an overview, FIG. 9A illustrates an example notification graphical user interface including a failure mode notification in accordance with one or more embodiments. By contrast, FIG. 9B illustrates an example error-pattern-analysis graphical user interface providing additional analysis for information from a failure mode notification.

In particular, FIG. 9A illustrates a notification graphical user interface 904 on a screen 902 of a user client device 900 (e.g., the user client device 108). The notification graphical user interface 904 includes a failure mode notification 906 comprising a failure mode element 908, a probability element 910, and a variation source graph element 912.

As illustrated in FIG. 9A, the failure mode notification 906 includes the failure mode element 908. The failure mode element 908 indicates one or more sequencing pipeline materials that the variation-source-identification system 106 has identified as potential failure modes. In some embodiments, the variation-source-identification system 106 determines a threshold number of potential failure sources to display within the failure mode element 908. For example, the variation-source-identification system 106 determines to display no more than three potential failure sources. In one or more embodiments, the variation-source-identification system 106 determines the threshold number of potential failure sources based on a threshold percent likelihood. In at least one example, the variation-source-identification system 106 determines to display potential failure sources having percent assignable cause variations over a probability threshold value. To illustrate, the variation-source-identification system 106 determines to display failure sources associated with percent assignable cause variations equal to or greater than 3%. In addition or in the alternative to text describing a potential failure source, in certain embodiments, the variation-source-identification system 106 generates and provides an error code for display on the notification graphical user interface 904—thereby indicating a failure source with a code.

As further shown in FIG. 9A, the failure mode notification 906 also includes the probability element 910. The probability element 910 indicates probabilities that the corresponding sequencing pipeline material is the failure source for a base-call-error type corresponding to a sequencing pipeline. In some embodiments, the probability element 910 equals the determined percent assignable cause variation.

FIG. 9A illustrates further the failure mode notification 906 including the variation source graph element 912. Based on detecting user interaction with the variation source graph element 912, in some embodiments, the user client device 900 updates the notification graphical user interface 904 to display a graph indicating percent assignable cause variations. In certain implementations, the variation-source-identification system 106 provides, for display via the notification graphical user interface 904, the graph illustrated in FIG. 6B. Additionally, or alternatively, the variation-source-identification system 106 selects specific bars from the graph illustrated in FIG. 6B to display via the notification graphical user interface 904. In particular, the variation-source-identification system 106 determines to display bars corresponding to the specific base-call-error types and/or neighboring nucleotide bases with base-call-error rates. The variation-source-identification system 106 can provide various types of graphs and visuals based on user selection of the variation source graph element 912. For example, the variation-source-identification system 106 may also present the graph illustrated in FIG. 3.

In some embodiments, the variation-source-identification system 106 provides, within the failure mode notification 906, an element to confirm a failure source. In particular, the user client device 900 may present the failure mode notification 906 and detect a user selection confirming a manufacturing material identified in the failure mode notification 906. For instance, the user can check the barrel pump cartridge and confirm, via selecting a selectable option on the user client device 900, the presence of a bubble or other malfunction within the barrel pump cartridge. In some embodiments, the failure mode notification 906 includes a selectable option to confirm a predicted failure source. For example, the failure mode notification 906 can include an option to confirm a barrel pump cartridge failure source. In another example, the failure mode notification 906 includes several selectable options each associated with a different failure source. For example, the failure mode notification 906 can include selectable options associated with each of the barrel pump cartridge, the well plate cartridge, and reagent 1. The variation-source-identification system 106 can confirm the presence of given failure source based on user selection of the given failure source. As mentioned previously, the variation-source-identification system 106 can further modify parameters of a machine learning model based on user interaction with the element to confirm the failure source.

In some embodiments, the variation-source-identification system 106 provides the failure mode notification 906 for display in real time (or near-real time) upon detecting a base-call-error pattern. Thus, the variation-source-identification system 106 can timely provide notice that a given sequencing material is likely causing a failure within the sequencing pipeline.

As mentioned, FIG. 9B illustrates an example error-pattern-analysis graphical user interface including additional information from a failure mode notification. In particular, FIG. 9B illustrates an error-pattern-analysis graphical user interface 914 on the screen 902 of the user client device 900. In particular, the error-pattern-analysis graphical user interface 914 includes a sequencing run element 916, a visualization modification element 918, a variables element 920, and an error visualization element 922. Generally, the error-pattern-analysis graphical user interface 914 provides a visualization of base-call-error patterns. In some embodiments, the variation-source-identification system 106 provides the error-pattern-analysis graphical user interface 914 for display based on receiving an indication of user selection of the variation source graph element 912 illustrated in FIG. 9A. In other embodiments, the variation-source-identification system 106 provides the error-pattern-analysis graphical user interface 914 based on user selection of an additional user interface element not illustrated in FIG. 9A.

FIG. 9B illustrates the error-pattern-analysis graphical user interface 914 including the error visualization element 922. By providing the error visualization element 922, the variation-source-identification system 106 generates a graphical visualization of a base-call-error pattern for one or more sequencing runs. For example, the error visualization element 922 illustrated in FIG. 9B includes box plots indicating an overall error rate (error rate) and patterns within correct calls organized by base. As illustrated, the error visualization element 922 includes indications of correct A calls (A A), correct C calls (C C), correct G calls (G G), and correct T calls (T T).

In other embodiments, the error visualization element 922 displays base-call-error rates organized according to base-call-error type. For example, the error visualization element 922 can include A→C base call errors, C→T base call errors, etc. Furthermore, the error visualization element 922 can include various types of visualizations. For example, and as mentioned, the error visualization element 922 can include box plots, bar graphs, column graphs, histograms, line graphs, scatter plots, and other types of graphs or charts.

As further illustrated in FIG. 9B, the error-pattern-analysis graphical user interface 914 includes the sequencing run element 916. The sequencing run element 916 indicates one or more sequencing runs portrayed by the error visualization element 922. For example, and as illustrated in FIG. 9B, the variation-source-identification system 106 can receive from the user client device 900 an indication of user interaction with a sequencing run listed in the sequencing run element 916. The user client device 900 can update the sequencing run element 916 to indicate the selected sequencing run, for example, by highlighting the selected sequencing run.

In addition to the sequencing run element 916, the error-pattern-analysis graphical user interface 914 also includes the variables element 920. In particular, the variables element 920 indicates variables visualized within the error visualization element 922. To illustrate, based on indications of user interactions with the variables element 920 from the user client device 900, the variation-source-identification system 106 can determine to visualize errors based on base-call-error type and flanking nucleotide bases. For instance, as illustrated in FIG. 9B, the user client device 900 receives data indicating user selection of a correct C→C base call when flanked by C_A. Based on detecting such a user selection, the user client device 900 can update the error visualization element 922 to include a visualization of the selected base-call-error type and flanking nucleotide bases.

In addition to the variables element 920, the error-pattern-analysis graphical user interface 914 further includes the visualization modification element 918. Based on user interaction with the visualization modification element 918, for instance, the user client device 900 can customize the visualization displayed within the error visualization element 922. In particular, the visualization modification element 918 includes, for each of the charts displayed within the error visualization element 922, a jitter modification element, an outliers element, a box type element, a box style element, a 5-number summary element, a response axis element, and a variables indication element. Based on user interaction with any of the elements within the visualization modification element 918, the user client device 900 can customize the error visualization element 922. For example, by deselecting the outliers element, the user client device 900 can remove all outliers from the error visualization element 922. In another example, the user client device 900 can update the error visualization element 922 to include other types of graphs and charts based on detected user interaction with the visualization modification element 918.

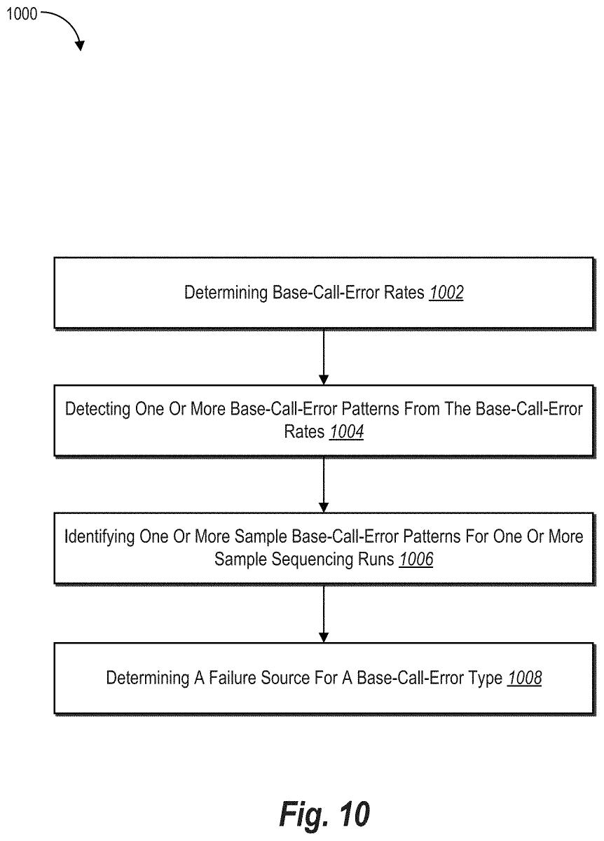

FIGS. 1-9B, the corresponding text, and the examples provide a number of different methods, systems, devices, and non-transitory computer-readable media of the variation-source-identification system 106. In addition to the foregoing, one or more embodiments can also be described in terms of flowcharts comprising acts for accomplishing a particular result, such as the flowchart of acts shown in FIG. 10. Additionally, the acts described herein may be repeated or performed in parallel with one another or in parallel with different instances of the same or similar acts.

FIG. 10 illustrates a flowchart of a series of acts 1000 for determining a failure source for a base-call-error type. While FIG. 10 illustrates acts according to one embodiment, alternative embodiments may omit, add to, reorder and/or modify any of the acts shown in FIG. 10. The acts of FIG. 10 can be performed as part of a method. Alternatively, a non-transitory computer-readable medium can comprise instructions that, when executed by one or more processors, cause a computing device to perform the acts of FIG. 10. In some embodiments, a system can perform the acts of FIG. 10.

In one or more embodiments, the series of acts 1000 is implemented on one or more computing devices, such as the computing device illustrated in FIG. 11. In addition, in some embodiments, the series of acts 1000 is implemented in a digital environment for sequencing nucleic-acid polymers. As illustrated in FIG. 10, the series of acts 1000 includes an act 1002 of determining base-call-error rates, an act 1004 of determining a base-call-error pattern from the base-call-error rates, an act 1006 of identifying a sample base-call-error-pattern for one or more sample sequencing runs, and an act 1008 of determining a failure source for a base-call-error type.

The series of acts 1000 illustrated in FIG. 10 includes the act 1002 of determining base-call-error rates. In particular, the act 1002 comprises determining base-call-error rates at which nucleotide-base calls generated by a sequencing pipeline differ from reference bases in a reference genome. In some embodiments the act 1002 further comprises determining the base-call-error rates by determining nucleotide-specific error rates at which nucleotide-base calls generated by the sequencing pipeline differ from the reference bases. In one or more embodiments, the act 1002 further comprises determining the base-call-error rates by utilizing a confusion matrix. In some embodiments, the act 1002 further comprises determining the base-call-error rates by normalizing a confusion matrix comprising base-call-error data based on a total of correct nucleotide-base calls for a specific type of nucleotide-base call. Additionally, in some embodiments, the act 1002 further comprises normalizing a confusion matrix comprising base-call-error data based on a total of correct nucleotide-base calls for a specific type of nucleotide-base call and one or more of cycle, time, or nucleotide read for a base-call error.

The series of acts 1000 includes the act 1004 of detecting one or more base-call-error patterns from the base-call-error rates grouped according to base-call-error types. In particular, the act 1004 comprises detecting a base-call-error pattern from the base-call-error rates grouped according to base-call-error types. In some embodiments, the act 1004 comprises determine the base-call-error rates grouped according to the base-call-error types and different neighboring nucleotide bases respectively flanking incorrect nucleotide-base calls; and detecting the one or more base-call-error patterns from the base-call-error rates grouped according to the base-call-error types and the different neighboring nucleotide bases.

The series of acts 1000 includes the act 1006 of identifying one or more sample base-call-error patterns for one or more sample sequencing runs. In particular, the act 1006 comprises based on the base-call-error pattern, based on the one or more base-call-error patterns, identifying one or more sample base-call-error patterns for one or more sample sequencing runs that utilize one or more sequencing pipelines corresponding to the sequencing pipeline. In some embodiments, the act 1006 comprises identify the one or more sample base-call-error patterns for the one or more sample sequencing runs by: categorizing sets of sample sequencing runs from sample sequencing runs that utilize similar manufacturing materials based on manufacturing identification data; detecting different sample base-call-error patterns for the sets of sample sequencing runs; and identifying the one or more sample base-call-error patterns from among the different sample base-call-error patterns for the sets of sample sequencing runs based on the correlation between the one or more base-call-error patterns and the one or more sample base-call-error patterns. Additionally, the act 1006 can further comprise detecting the different sample base-call-error patterns by: aggregating sample nucleotide-fragment reads for the sample sequencing runs; determining sample nucleotide-specific error rates at which the sample nucleotide-base calls differ from the reference bases; and grouping the sample nucleotide-specific error rates according to the base-call-error types and different neighboring nucleotide bases respectively flanking incorrect nucleotide-base calls. In some embodiments, the act 1006 further comprises categorizing the sets of sample sequencing runs that utilize similar manufacturing materials by: truncating the manufacturing identification data; and generating a set of sequencing runs by grouping a threshold number of sequencing runs that share a same truncated manufacturing identification data.

Additionally, in some embodiments, the act 1006 further comprises identifying the one or more sample base-call-error patterns for the one or more sample sequencing runs by identifying an existing sample base-call-error pattern for the one or more sample sequencing runs or detecting a new sample base-call-error pattern for the one or more sample sequencing runs.

As further illustrated in FIG. 10, the series of acts 1000 also includes the act 1008 of determining a failure source for a base-call-error type. In particular, the act 1008 comprises based on a correlation between the one or more base-call-error patterns and the one or more sample base-call-error patterns, determining a failure source for a base-call-error type corresponding to the sequencing pipeline. In some embodiments, the act 1008 comprises based on a probability of the one or more base-call-error patterns corresponding to the one or more sample base-call-error patterns, determining a failure source for a base-call-error type corresponding to the sequencing pipeline. In some embodiments, the act 1008 further comprises determining the failure source corresponding to the sequencing pipeline by determining contribution metrics indicating contributions of sequencing-pipeline materials to base-call errors from the sequencing pipeline; and determining the failure source for the base-call-error type based on the contribution metrics. Additionally, in some embodiments, the act 1008 further comprises determining the contribution metrics by determining assignable cause variations for the sequencing-pipeline materials contributing to the base-call errors from the sequencing pipeline. In some embodiments, the act 1008 further comprises determining the failure source by identifying a consumable product, a part of a sequencing machine, a software application or feature, or a part of a nucleotide-sample slide as a contributing factor to a sequencing variation in the sequencing pipeline.

In some embodiments, the act 1008 further comprises determining the failure source corresponding to the sequencing pipeline by: determining, utilizing a statistical model, contribution metrics indicating probabilities of sequencing-pipeline materials contributing to base-call errors from the sequencing pipeline; and determining the failure source for the base-call-error type based on the contribution metrics. Furthermore, the act 1008 can comprise determining the contribution metrics utilizing the statistical model by utilizing a variance components model to generate percentages of assignable cause variations for the sequencing-pipeline materials contributing to the base-call errors. In some embodiments, the act 1008 comprises determining the correlation between the one or more base-call-error patterns and the one or more sample base-call-error patterns by utilizing a variance components model to determine percentages of assignable cause variations for sequencing-pipeline materials contributing to base-call errors of the base-call-error type.

In some embodiments, the series of acts 1000 includes an additional act of providing, for display on a computing device associated with the sequencing pipeline, a notification indicating the failure source.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic-acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

The SBS techniques described below can utilize single-read sequencing or paired-end sequencing. In single-rea sequencing, the sequencing device reads a fragment from one end to another to generate the sequence of base pairs. In contrast, during paired-end sequencing, the sequencing device begins at one read, finishes reading a specified read length in the same direction, and begins another read from the opposite end of the fragment.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g. A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such embodiments, each image will show nucleic acid features that have incorporated nucleotides of a particular type. Different features will be present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features will be present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

An advantage of the methods set forth herein is that they provide for rapid and efficient detection of a plurality of target nucleic acid in parallel. Accordingly the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, Calif.) and devices described in U.S. Ser. No. 13/273,666, which is incorporated herein by reference.

The sequencing system described above sequences nucleic-acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the variation-source-identification system 106 can include software, hardware, or both. For example, the components of the variation-source-identification system 106 can include one or more instructions stored on a non-transitory computer readable storage medium and executable by processors of one or more computing devices (e.g., the user client device 108). When executed by the one or more processors, the computer-executable instructions of the variation-source-identification system 106 can cause the computing devices to perform the failure source identification methods described herein. Alternatively, the components of the variation-source-identification system 106 can comprise hardware, such as special purpose processing devices to perform a certain function or group of functions. Additionally, or alternatively, the components of the variation-source-identification system 106 can include a combination of computer-executable instructions and hardware.

Furthermore, the components of the variation-source-identification system 106 performing the functions described herein with respect to the variation-source-identification system 106 may, for example, be implemented as part of a stand-alone application, as a module of an application, as a plug-in for applications, as a library function or functions that may be called by other applications, and/or as a cloud-computing model. Thus, components of the variation-source-identification system 106 may be implemented as part of a stand-alone application on a personal computing device or a mobile device. Additionally, or alternatively, the components of the variation-source-identification system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer readable medium and executable by one or more computing devices (e.g., any of the media content access devices described herein). In general, a processor (e.g., a microprocessor) receives instructions, from a non-transitory computer readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a MC), and then eventually transferred to computer system RANI and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 11 illustrates a block diagram of a computing device 1100 that may be configured to perform one or more of the processes described above. One will appreciate that one or more computing devices such as the computing device 1100 may implement the variation-source-identification system 106 and the sequencing system 104. As shown by FIG. 11, the computing device 1100 can comprise a processor 1102, a memory 1104, a storage device 1106, an I/O interface 1108, and a communication interface 1111, which may be communicatively coupled by way of a communication infrastructure 1111. In certain embodiments, the computing device 1100 can include fewer or more components than those shown in FIG. 11. The following paragraphs describe components of the computing device 1100 shown in FIG. 11 in additional detail.

In one or more embodiments, the processor 1102 includes hardware for executing instructions, such as those making up a computer program. As an example, and not by way of limitation, to execute instructions for dynamically modifying workflows, the processor 1102 may retrieve (or fetch) the instructions from an internal register, an internal cache, the memory 1104, or the storage device 1106 and decode and execute them. The memory 1104 may be a volatile or non-volatile memory used for storing data, metadata, and programs for execution by the processor(s). The storage device 1106 includes storage, such as a hard disk, flash disk drive, or other digital storage device, for storing data or instructions for performing the methods described herein.

The I/O interface 1108 allows a user to provide input to, receive output from, and otherwise transfer data to and receive data from computing device 1100. The I/O interface 1108 may include a mouse, a keypad or a keyboard, a touch screen, a camera, an optical scanner, network interface, modem, other known I/O devices or a combination of such I/O interfaces. The I/O interface 1108 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1108 is configured to provide graphical data to a display for presentation to a user. The graphical data may be representative of one or more graphical user interfaces and/or any other graphical content as may serve a particular implementation.

The communication interface 1111 can include hardware, software, or both. In any event, the communication interface 1111 can provide one or more interfaces for communication (such as, for example, packet-based communication) between the computing device 1100 and one or more other computing devices or networks. As an example, and not by way of limitation, the communication interface 1111 may include a network interface controller (MC) or network adapter for communicating with an Ethernet or other wire-based network or a wireless MC (WNIC) or wireless adapter for communicating with a wireless network, such as a WI-FI.

Additionally, the communication interface 1111 may facilitate communications with various types of wired or wireless networks. The communication interface 1111 may also facilitate communications using various communication protocols. The communication infrastructure 1111 may also include hardware, software, or both that couples components of the computing device 1100 to each other. For example, the communication interface 1111 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer readable medium comprising instructions that, when executed by the at least one processor, cause the system to:
determine base-call-error rates at which nucleotide-base calls generated by a sequencing pipeline differ from reference bases in a reference genome;
detect one or more base-call-error patterns from the base-call-error rates grouped according to base-call-error types;
based on the one or more base-call-error patterns, identify one or more sample base-call-error patterns for one or more sample sequencing runs that utilize one or more sequencing pipelines corresponding to the sequencing pipeline; and
based on a correlation between the one or more base-call-error patterns and the one or more sample base-call-error patterns, determine a failure source for a base-call-error type corresponding to the sequencing pipeline.

2. The system of claim 1, further comprising instructions that, when executed by the at least one processor, cause the system to determine the base-call-error rates by determining nucleotide-specific error rates at which nucleotide-base calls generated by the sequencing pipeline differ from the reference bases.

3. The system of claim 1, further comprising instructions that, when executed by the at least one processor, cause the system to:
determine the base-call-error rates grouped according to the base-call-error types and different neighboring nucleotide bases respectively flanking incorrect nucleotide-base calls; and
detect the one or more base-call-error patterns from the base-call-error rates grouped according to the base-call-error types and the different neighboring nucleotide bases.

4. The system of claim 1, further comprising instructions that, when executed by the at least one processor, cause the system to determine the failure source corresponding to the sequencing pipeline by:
determining contribution metrics indicating contributions of sequencing-pipeline materials to base-call errors from the sequencing pipeline; and
determining the failure source for the base-call-error type based on the contribution metrics.

5. The system of claim 4, further comprising instructions that, when executed by the at least one processor, cause the system to determine the contribution metrics by determining assignable cause variations for the sequencing-pipeline materials contributing to the base-call errors from the sequencing pipeline.

6. The system of claim 1, further comprising instructions that, when executed by the at least one processor, cause the system to provide, for display on a computing device associated with the sequencing pipeline, a notification indicating the failure source.

7. The system of claim 1, further comprising instructions that, when executed by the at least one processor, cause the system to determine the failure source by identifying a consumable product, a part of a sequencing machine, a software application or feature, or a part of a nucleotide-sample slide as a contributing factor to a sequencing variation in the sequencing pipeline.

8. The system of claim 1, further comprising instructions that, when executed by the at least one processor, cause the system to determine the base-call-error rates by utilizing a confusion matrix.

9. The system of claim 1, further comprising instructions that, when executed by the at least one processor, cause the system to identify the one or more sample base-call-error patterns for the one or more sample sequencing runs by:
categorizing sets of sample sequencing runs from sample sequencing runs that utilize similar manufacturing materials based on manufacturing identification data;
detecting different sample base-call-error patterns for the sets of sample sequencing runs; and
identifying the one or more sample base-call-error patterns from among the different sample base-call-error patterns for the sets of sample sequencing runs based on the correlation between the one or more base-call-error patterns and the one or more sample base-call-error patterns.

10. The system of claim 9, further comprising instructions that, when executed by the at least one processor, cause the system to detect the different sample base-call-error patterns by:
aggregating sample nucleotide-fragment reads for the sample sequencing runs;
determining sample nucleotide-specific error rates at which the sample nucleotide-base calls differ from the reference bases; and
grouping the sample nucleotide-specific error rates according to the base-call-error types and different neighboring nucleotide bases respectively flanking incorrect nucleotide-base calls.

11. The system of claim 9, further comprising instructions that, when executed by the at least one processor, cause the system to categorize the sets of sample sequencing runs that utilize similar manufacturing materials by:
truncating the manufacturing identification data; and
generating a set of sequencing runs by grouping a threshold number of sequencing runs that share a same truncated manufacturing identification data.

12. A non-transitory computer readable medium comprising instructions that, when executed by at least one processor, cause a computing device to:
determine base-call-error rates at which nucleotide-base calls generated by a sequencing pipeline differ from reference bases in a reference genome;
detect one or more base-call-error patterns from the base-call-error rates grouped according to base-call-error types;
based on the one or more base-call-error patterns, identify one or more sample base-call-error patterns for one or more sample sequencing runs that utilize one or more sequencing pipelines corresponding to the sequencing pipeline; and
based on a probability of the one or more base-call-error patterns corresponding to the one or more sample base-call-error patterns, determine a failure source for a base-call-error type corresponding to the sequencing pipeline.

13. The non-transitory computer readable medium of claim 12, further comprising instructions that, when executed by the at least one processor, cause the computing device to determine the failure source corresponding to the sequencing pipeline by:
determining, utilizing a statistical model, contribution metrics indicating probabilities of sequencing-pipeline materials contributing to base-call errors from the sequencing pipeline; and
determining the failure source for the base-call-error type based on the contribution metrics.

14. The non-transitory computer readable medium of claim 13, further comprising instructions that, when executed by the at least one processor, cause the computing device to determine the contribution metrics utilizing the statistical model by utilizing a variance components model to generate percentages of assignable cause variations for the sequencing-pipeline materials contributing to the base-call errors.

15. The non-transitory computer readable medium of claim 12, further comprising instructions that, when executed by the at least one processor, cause the computing device to identify the one or more sample base-call-error patterns for the one or more sample sequencing runs by identifying an existing sample base-call-error pattern for the one or more sample sequencing runs or detecting a new sample base-call-error pattern for the one or more sample sequencing runs.

16. The non-transitory computer readable medium of claim 12, The non-transitory computer readable medium of claim 12, further comprising instructions that, when executed by the at least one processor, cause the computing device to determine the base-call-error rates by normalizing a confusion matrix comprising base-call-error data based on a total of correct nucleotide-base calls for a specific type of nucleotide-base call.

17. A computer-implemented method comprising:
determining base-call-error rates at which nucleotide-base calls generated by a sequencing pipeline differ from reference bases in a reference genome;
detecting one or more base-call-error patterns from the base-call-error rates grouped according to base-call-error types;
based on the one or more base-call-error patterns, identifying one or more sample base-call-error patterns for one or more sample sequencing runs that utilize one or more sequencing pipelines corresponding to the sequencing pipeline; and
based on a correlation between the one or more base-call-error patterns and the one or more sample base-call-error patterns, determining a failure source for a base-call-error type corresponding to the sequencing pipeline.

18. The computer-implemented method of claim 17, further comprising:
determining the base-call-error rates grouped according to different neighboring nucleotide bases flanking incorrect nucleotide-base calls; and
detecting the one or more base-call-error patterns from the base-call-error rates grouped according to the different neighboring nucleotide bases.

19. The computer-implemented method of claim 17, wherein determining the base-call-error rates comprises normalizing a confusion matrix comprising base-call-error data based on a total of correct nucleotide-base calls for a specific type of nucleotide-base call and one or more of cycle, time, or nucleotide read for a base-call error.

20. The computer-implemented method of claim 17, further comprising determining the correlation between the one or more base-call-error patterns and the one or more sample base-call-error patterns by utilizing a variance components model to determine percentages of assignable cause variations for sequencing-pipeline materials contributing to base-call errors of the base-call-error type.
